(19) United States
(12) Patent Application Publication
(10) Pub. No.: US 2026/0347346 A1
(43) Pub. Date: Nov. 5, 2026

(54) SUBSTITUTED 6-(PYRIMIDIN-4-YL)QUINOLINE COMPOUNDS AS CYCLIN DEPENDENT KINASE INHIBITORS

(71) Applicant: BeOne Medicines I GmbH, Basel (CH)

(72) Inventors: Jing LI, Beijing (CN); Wenqing XU, Beijing (CN); Zhiwei WANG, Beijing (CN)

(73) Assignee: BeOne Medicines I GmbH, Basel (CH)

(21) Appl. No.: 19/183,988

(22) Filed: Apr. 21, 2025

Related U.S. Application Data
(63) Continuation of application No. PCT/CN2023/126591, filed on Oct. 25, 2023.

(30) Foreign Application Priority Data
Oct. 27, 2022 (WO) PCT/CN2022/127942

(51) Int. Cl. C07D 405/14; A61K 31/506; C07D 401/14

(52) U.S. Cl. CPC C07D 405/14; A61K 31/506; C07D 401/14

(57) ABSTRACT

This disclosure provides compounds containing 6-(pyrimidin-4-yl)quinoline structure, the use thereof for selectively inhibiting the activity of CDK4, and pharmaceutical compositions comprising the compounds as treatment of various diseases including cancer.

### CROSS-REFERENCE TO RELATED APPLICATIONS

This application is a continuation of International Application No. PCT/CN2023/126591, filed on Oct. 25, 2023, which claims priority to International Application No. PCT/CN2022/127942, filed on Oct. 27, 2022, the disclosures of each of which are hereby incorporated by reference in their entireties.

### FIELD OF THE INVENTION

This disclosure provides compounds containing 6-(pyrimidin-4-yl) quinoline structure, the use thereof for selectively inhibiting the activity of cyclin-dependent kinase 4 (CDK4), and pharmaceutical compositions comprising the compounds as the treatment of various diseases including cancer.

### BACKGROUND OF THE INVENTION

Human kinase is a large group of enzymes that add phosphate groups (PO₄³⁻) to other molecules in human body [1. FASEB J. 1995 May; 9 (8): 576-96. 2. Enzyme Res. 2011; 2011:794089.]. There are more than 500 kinase-encoding genes that exist in the human genome and their substrates including proteins, lipids, and nucleic acids [3. Cell Signal. 2004 September; 16 (9): 983-9. 4. Cell. 2017 Aug. 10; 170 (4): 605-635.]. Kinase mis-regulation is identified in many diseases including cancer, autoimmunity, neurological disorders, diabetes and cardiovascular disease. For example, the mutated kinases can become constitutively active and thus cause diverse cellular anomalies, leading to cancer initiation or growth. Using small molecular inhibitors to inhibit kinase activity is proved to be a successful method to treat cancer and other disease [5. Expert Rev Anticancer Ther. 2018 December; 18 (12): 1249-1270.]. Up to now, there are more than 70 kinase inhibitors have been approved by FDA, EMA or CDE as drugs [6. Nat Rev Drug Discov. 2018 May; 17 (5): 353-377.].

Protein kinase family takes a majority fraction of the kinase superfamily. For protein targets, protein kinases can phosphorylate the amino acids including serine, threonine, tyrosine and histidine. [7. Science. 2002 Dec. 6; 298 (5600): 1912-34.] Protein kinases play a major role in cellular activation processes, through reversible phosphorylation and dephosphorylation of proteins, by the antagonistic action of kinases and phosphatases, is an important component of cell signaling because the phosphorylated and unphosphorylated states of the target protein can have different levels of activity. [8. Biochimie. 2014 December; 107 Pt B: 167-87. 9. Clin Transl Oncol. 2006 March; 8 (3): 153-60.] Different protein kinases including EGFR, BTK, ALK, JAK, PI3K and CDK are proved to be good targets for cancer drug development.

Excessively activated cell cycle is a common feature of human cancer [10. Nat Rev Cancer. 2009 March; 9 (3): 153-66.]. While cyclins are among the most important core cell cycle regulators. There are four basic cyclin types found in humans including G1 cyclins, G1/S cyclins, S cyclins and M cyclins. To drive the cell cycle forward, a cyclin must activate or inactivate many target proteins inside of the cell. And these cyclins drive the events of the cell cycle majorly by partnering with a family of enzymes called the cyclin-dependent kinases (Cdks). Cdk kinase itself is inactive, but binding with a cyclin can activate it, making the CDK/cyclin complex a functional holoenzyme and allowing it to modify target proteins [11. Orphanet J Rare Dis. 2020 Aug. 6; 15 (1): 203. 12. J Mol Biol. 1999 Apr. 16; 287 (5): 821-8.]. There are 26 serine/threonine protein kinases that form a CDK and CDK-like branch of the CMGC subfamily of the human kinome; of these, 21 are classified as CDKs. Among all the currently identified CDKs, CDK1, CDK2, CDK4 and CDK6 are considered as the direct modulate of cell cycle majorly by phosphorylating and inactivating RB protein and releasing E2F transcription factors, and E2F downstream pathway is critical in regulating the initiation of DNA replication. And CDK4/6 is essential for G1 early initiation and G1/S transition. [13. Cell Death Differ. 1998 February; 5 (2): 132-40. 14. Oncogene. 2016 Sep. 15; 35 (37): 4829-35.]

CDK4/6 related pathway is commonly deregulated in many different cancer types such as breast cancer, lung cancer and pancreatic cancer. And there are 4 approved CDK4/6 inhibitors including palbociclib, ribociclib, abemaciclib and trilaciclib which have been approved by FDA or CDE to be used as either single agent or combo with endocrine therapy to treat HR+, Her2− breast cancer. This approach shows good efficacy in clinic while CDK4/6 inhibitors more or less lead to hemopoietic toxicity like neutropenia and leukopenia which highly limits the clinical application of CDK4/6 inhibitors. And emerging data indicating inhibition of CDK6/Cyclin D3 may cause the clinical observed hematologic toxicity [15. Cell. 2004 Aug. 20; 118 (4): 493-504. 16. Haematologica. 2021 Oct. 1; 106 (10): 2624-2632.] while CDK4/Cyclin D1 is the oncogenic driver in different cancers [17. Nat Commun. 2019 Dec. 20; 10 (1): 5817. 18. 18. Cancer Cell. 2006 January; 9 (1): 23-32.]. Developing a CDK4 selective inhibitor might lead to advantages including improved efficacy, mitigated hematologic toxicity and expanding clinical usage in many cancers including but not limited to breast cancer, lung cancer, pancreatic cancer, prostate cancer, bone cancer, liver cancer and endometrial cancer.

There remains a great need to develop a CDK4 selective inhibitor since the protein structure of CDK4 and CDK4 share very high homology. Most of the previously reported compounds are CDK4/6 dual inhibitors. Here we report compounds with high CDK4 selectivity over all other kinases including CDK6, which potentially lead to better efficacy, improved toxicity profile and potential to overcome resistance mechanisms, and the like.

### SUMMARY OF THE INVENTION

One objective of the present invention is to provide compounds and derivatives which function to act as CDK4 inhibitors, and methods of preparation and uses thereof.

Aspect 1. A compound of formula (I):

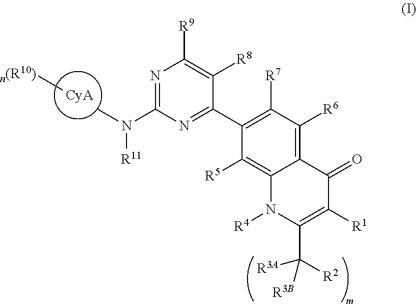

or a N-oxide thereof, or a pharmaceutically acceptable salt thereof, or a stereoisomer thereof, or a tautomer thereof, or a deuterated analog thereof, or a prodrug thereof,
wherein:
ring CyA is a 3- to 8-membered ring, said ring comprising 0, 1 or 2 heteroatom(s) independently selected from nitrogen, oxygen, or optionally oxidized sulfur as ring member(s); said ring is optionally substituted with at least one substituent R¹⁰;
n is 0, 1, 2, 3, 4 or 5;
m is 0 or 1; provided that when m=0, the

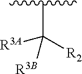

moiety as a whole is replaced with H;
R¹ is H, halogen, —C₁₋₈alkyl, —C₂₋₈alkenyl, —C₂₋₈alkynyl, cycloalkyl, haloalkyl, heterocyclyl, aryl, heteroaryl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent Rid;
R^{1a}, R^{1b} and R^{1c} are each independently selected from hydrogen, —C₁₋₈alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1d} and R^{1f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R² is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2d};
R^{2a}, R^{2b} and R^{2c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2f}; or
(R^{2a} and R^{2b}), (R^{2b} and R^{2c}) or (R^{2a} and R^{2e}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(two R^{2d}) or (two R^{2f}) together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2g}, R^{2h} and R²ⁱ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3A} and R^{3B} are each independently hydrogen, halogen, —C₁₋₈alkyl, —C₂₋₈alkenyl, —C₂₋₈alkynyl, cycloalkyl, heterocyclyl, aryl, heteroaryl or —CN; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3c}; or
R^{3A} and R^{3B} together with the atom to which they are attached, form acyl (—C(═O)—) or a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SOR^{3e}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3d}, R^{3e} and R^{3f} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁴ is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{401b}, —SO₂R^{401b}, —SO₂NR^{401b}R^{401c}, —COR^{401b}, —CO₂R^{401b}, —CONR^{401b}R^{401e}, —NO₂, —NR^{401b}R^{4c}, —NR^{401b}COR^{401c}, —NR^{401b}CO₂R^{401c}, —NR^{401b}CONR^{401c}R^{401d} or —NR^{401c}SO₂R^{401c}; wherein each of said —C₁₋₈alkyl, —C₂₋₈alkenyl, —C₂₋₈alkynyl, cycloalkyl heterocyclyl, aryl or heteroaryl is optionally substituted with at least one substituent R^{4a};
R^{401b}, R^{401c} and R^{401d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CON^{4c}R^{4d} or —NR^{4b}SO₂R⁴; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁴ is hydrogen, halogen, —C₁₋₈alkyl, —C₂₋₈alkenyl, —C₂₋₈alkynyl, cycloalkyl or heterocyclyl; wherein each of said —C₁₋₈alkyl, —C₂₋₈alkenyl, —C₂₋₈alkynyl, cycloalkyl or heterocyclyl is optionally substituted with at least one substituent R^{4a};
R^{4a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁵, R⁶, R⁷, R⁸ and R⁹ are each independently selected from H, halogen, —C₁₋₈alkyl, —C₂₋₈alkenyl, —C₂₋₈alkynyl, cycloalkyl, heterocyclyl, aryl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5d},
R^{5a}, R^{5b} and R^{5c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5f};
R^{5d} and R^{5f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹⁰ is selected from H, halogen, —C₁₋₈alkyl, —C₂₋₈alkenyl, —C₂₋₈alkynyl, cycloalkyl, heterocyclyl, aryl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10d}.
R^{10a}, R^{10b} and R^{10c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10f};
R^{10d} and R^{10f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹¹ is selected from H, —C₁₋₈alkyl, —C₂₋₈alkenyl, —C₂₋₈alkynyl; wherein each of said —C₁₋₈alkyl, —C₂₋₈ alkenyl or —C₂₋₈alkynyl is optionally substituted with at least one substituent R^{11a},
R^{11a} is selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

Aspect 2. The compound of Aspect 1, wherein the compound is selected from formula (IIa), (IIb), (IIc), (IId), (Ile) or (IIaa):

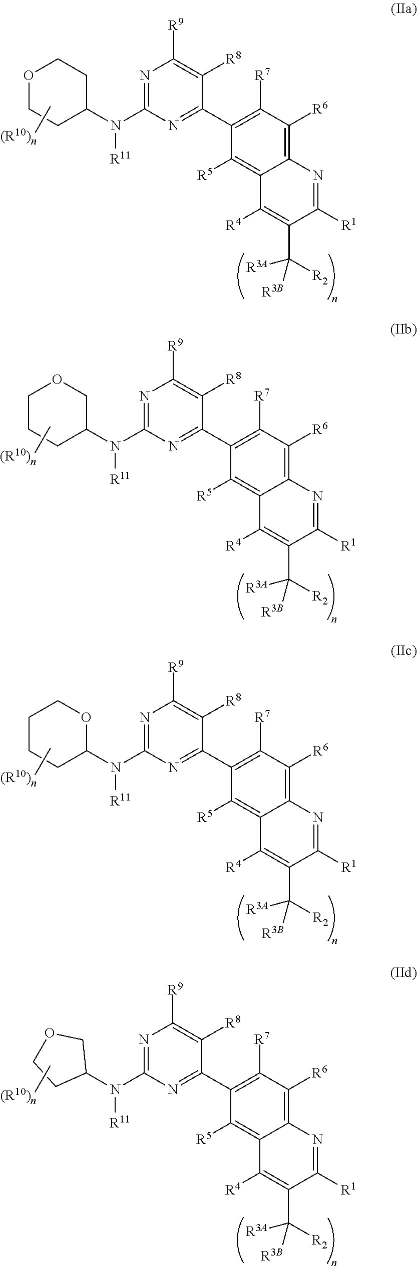

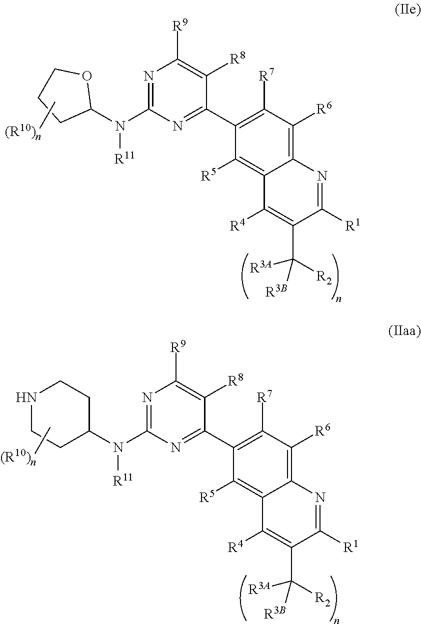

wherein, R¹, R², R^{3A}, R^{3B}, R⁴, R⁵, R⁶, R⁷, R⁸, R⁹, R¹⁰, R¹¹, m and n are each defined as Aspect 1;
preferably, the compound is selected from formula (IIf), (IIg), (Ilh), (IIi), (IIab) or (IIac):

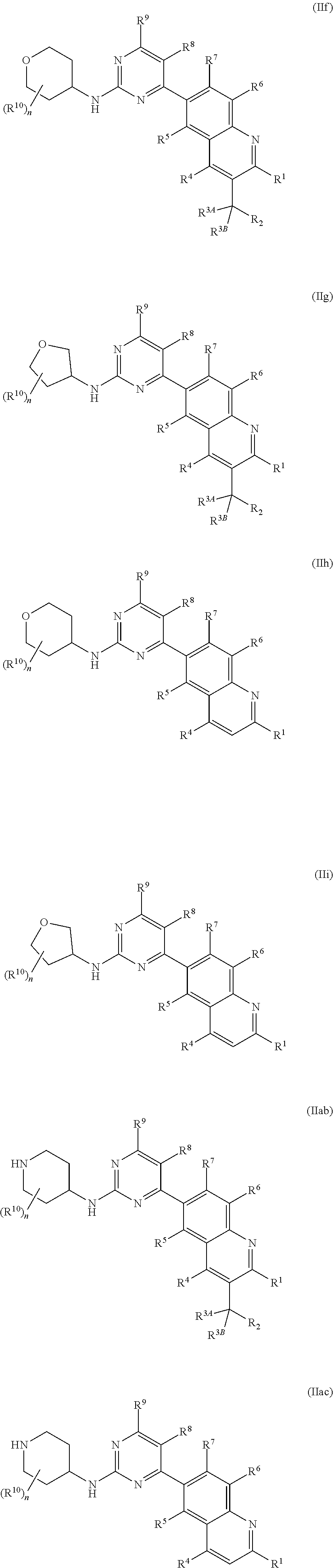

wherein, R¹, R², R^{3A}, R^{3B}, R⁴, R⁵, R⁶, R⁷, R⁸, R⁹, R¹⁰ and n are each defined as Aspect 1;
more preferably, the compound is selected from formula (IIj), (Ilk), (III) or (IIm):

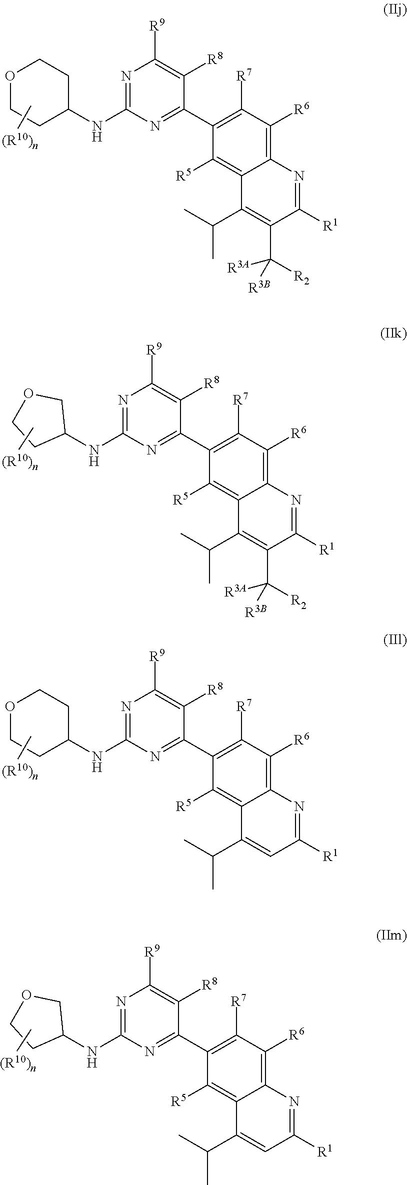

wherein, R¹, R², R^{3A}, R^{3B}, R⁵, R⁶, R⁷, R⁸, R⁹, R¹⁰ and n are each defined as Aspect 1;

even more preferably, the compound is selected from formula (IIn), (IIo), (IIp) or (IIq):

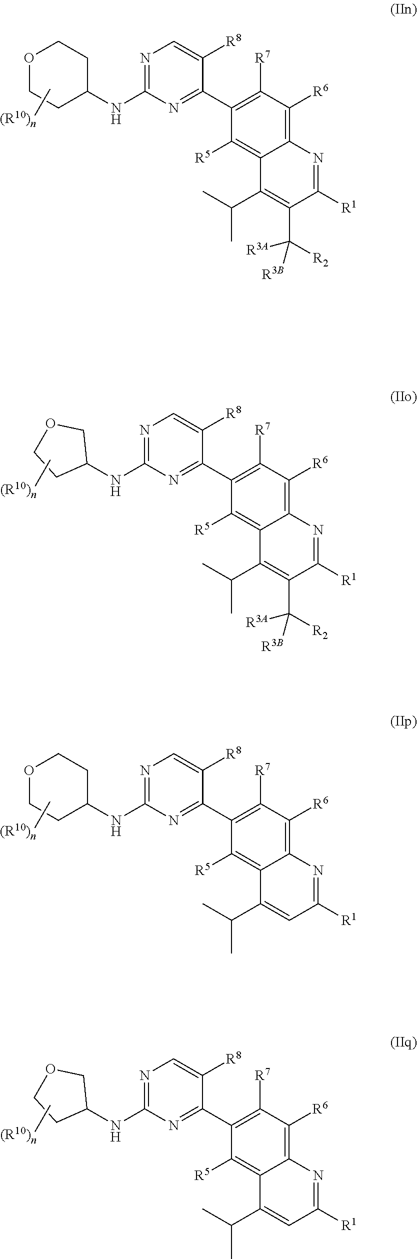

wherein, R¹, R², R^{3A}, R^{3B}, R⁵, R⁶, R⁷, R⁸, R¹⁰, m and n are each defined as Aspect 1.

Aspect 3. The compound of anyone of the preceding Aspects, wherein ring CyA is a 3-, 4-, 5-, 6-, 7- or 8-membered ring, said ring comprising 0, 1 or 2 heteroatom(s) independently selected from nitrogen, oxygen, or optionally oxidized sulfur as ring member(s); said ring is optionally substituted with 0, 1, 2, 3, 4 or 5 R¹⁰; said ring is a saturated or unsaturated ring;

preferably CyA is a 3-, 4-, 5-, 6-, 7- or 8-membered saturated ring, said ring comprising 0, 1 or 2 heteroatom(s) independently selected from nitrogen, oxygen, or optionally oxidized sulfur as ring member(s); said ring is optionally substituted with 0, 1, 2 or 3 R¹⁰;
more preferably CyA is a 5-, 6- or 7-membered saturated ring, said ring comprising 1 or 2 heteroatom(s) independently selected from nitrogen or oxygen as ring member(s); said ring is optionally substituted with 0, 1, 2 or 3 R¹⁰;
even more preferably, CyA is a ring selected from tetrahydrofuranyl or tetrahydropyranyl; said ring is optionally substituted with 0, 1, 2 or 3 R¹⁰; or
even more preferably, CyA is a ring selected from piperidinyl; said ring is optionally substituted with 0, 1, 2 or 3 R¹⁰.

Aspect 4. The compound of anyone of the preceding Aspects, wherein ring CyA is

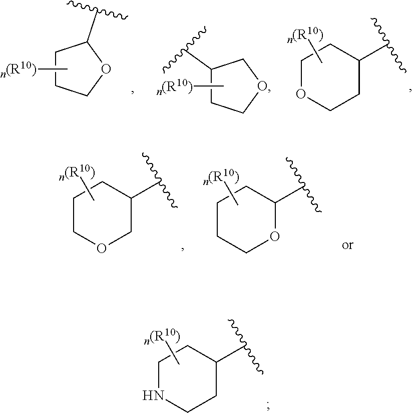

preferably, CyA is

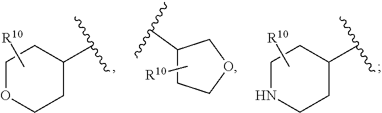

more preferably, CyA is

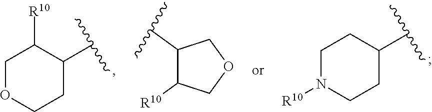

even more preferably, CyA is

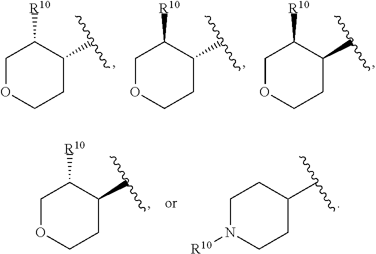

Aspect 5. The compound of anyone of the preceding Aspects, wherein R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said methyl, ethyl, propyl, butyl, pentyl, hexyl, heptyl, octyl, —C₂₋₈alkenyl, —C₂₋₈ alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10d}.
R^{10a}, R^{10b} and R^{10c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10f};
R^{10d} and R^{10f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OH, —NH₂, —COOMe, —COOEt, —COOCH₂CH₂CH₃, —COOCH(CH₃)₂, —COOCH₂CH₂CH₂CH₃, —COOCH(CH₃)CH₂CH₃, —COOCH₂CH(CH₃)₂ or —COOC(CH₃)₃;
more preferably, R¹⁰ is —OH, —COOMe, —COOEt, —COOCH₂CH₂CH₃, —COOCH(CH₃)₂, —COOCH₂CH₂CH₂CH₃, —COOCH(CH₃)CH₂CH₃, —COOCH₂CH(CH₃) 2 or —COOC(CH₃)₃.

Aspect 6. The compound of anyone of the preceding Aspects, wherein the

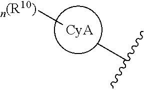

moiety is

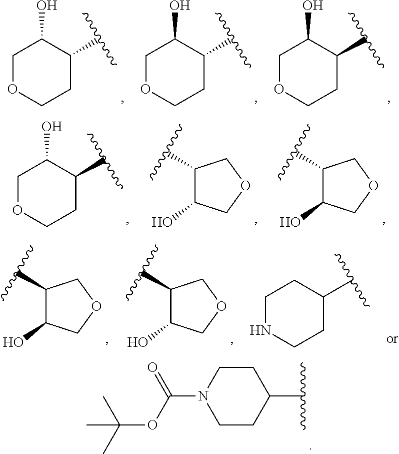

Aspect 7. The compound of anyone of the preceding Aspects, wherein R¹ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl, heterocyclyl, —CN, —OR^{1a}, —COR^{1a}, —CO₂R^{1a}, —CONR^{1a}R^{1b}, —NR^{1a}R^{1b}, —NR^{1a}CO₂R^{1b}, —NR^{1a}CO₂R^{1b} or —NR^{1a}CONR^{1b}R^{1c}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl or heterocyclyl is optionally substituted with at least one substituent R^{1d}.

R^{1a}, R^{1b} and R^{1e} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl is optionally substituted with at least one substituent R^{1f};

R^{1d} and R^{1f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, clopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl, heterocyclyl or —CN;
more preferably, R¹ is H, methyl, ethyl, n-propyl, iso-propyl, n-butyl, sec-butyl, iso-butyl or tert-butyl.

Aspect 8. The compound of anyone of the preceding Aspects, wherein R² is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2d};
R^{2a}, R^{2b} and R^{2c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 9. The compound of anyone of the preceding Aspects, wherein R² is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2a}, —COR^{2a}, —CO₂R^{2a}, —CONR^{2a}R^{2b}, —NR^{2a}R^{2b}, —NR^{2a}COR^{2b}; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d}.
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c] pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2g}, —SO₂R^{2g}, —COR^{2g}, —CO₂R², —CONR^{2g}R^{2h}, —NO₂, —NR^{2g}R^{2h} or —NR^{2g}COR^{2h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent-F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 10. The compound of anyone of the preceding Aspects, wherein R² is hydrogen, methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, —OR^{2a}, —NR^{2a}R^{2b} or —NR^{2a}COR^{2b}; wherein each of said methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c] pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d}.
R^{2a} and R^{2b} are each independently selected from hydrogen, methyl, ethyl, propyl (n-propyl or iso-propyl), butyl (n-butyl, sec-butyl, iso-butyl or tert-butyl), pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2g}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f},
R^{2d} and R^{2f} are each independently selected from hydrogen, —F, —Cl, —Br, —I, methyl, ethyl, propyl, butyl, pentyl, —CF₃, —CF₂H, —CFH₂, —CH₂CF₃, —CF₂CH₃, —CH₂OH, —CH(CH₃)OH, —C(CH₃)₂OH, —CH₂CH₂OH, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OH, methoxy, ethoxy, propoxy, butoxy, pentoxy, hexoxy, hepthoxy, octoxy, —SO₂Me, —SO₂Et, —SO₂C₃H₇, —COMe, —COEt, —COC₃H₇, —NH₂, —NHCH₃, —N(CH₃)₂, —NHC₂H₅, —NHC₃H₇, —NHC₄H₉, —CONH₂, —CONHCH₃, —CON(CH₃)₂, —CONHC₂H₅, —CONHC₃H₇, —CONHC₄H₉.

Aspect 11. The compound of anyone of the preceding Aspects, wherein R² is —H, -Me, —OMe, —OH, —NH₂, —NHCH₃, —N(CH₃)₂, —NHCH(CH₃)₂, —NHC(CH₃) 3, —NHCOCH₃,

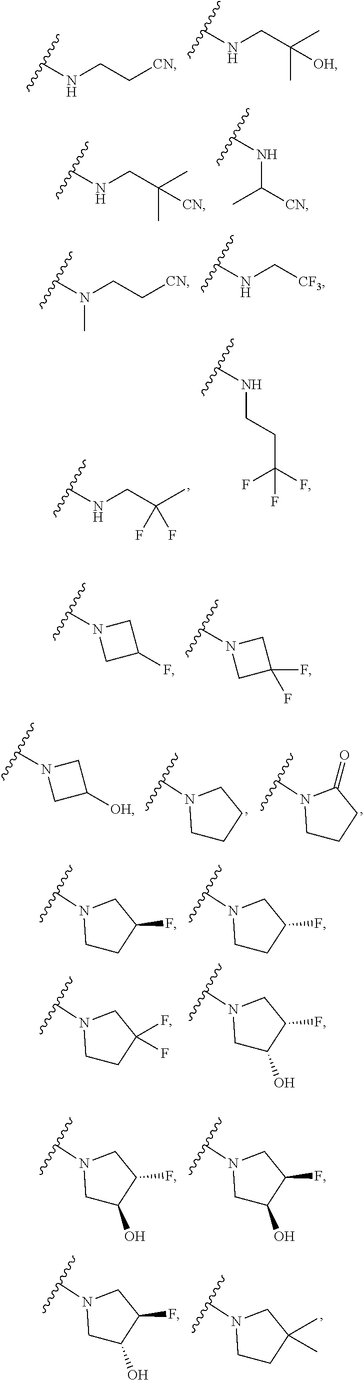

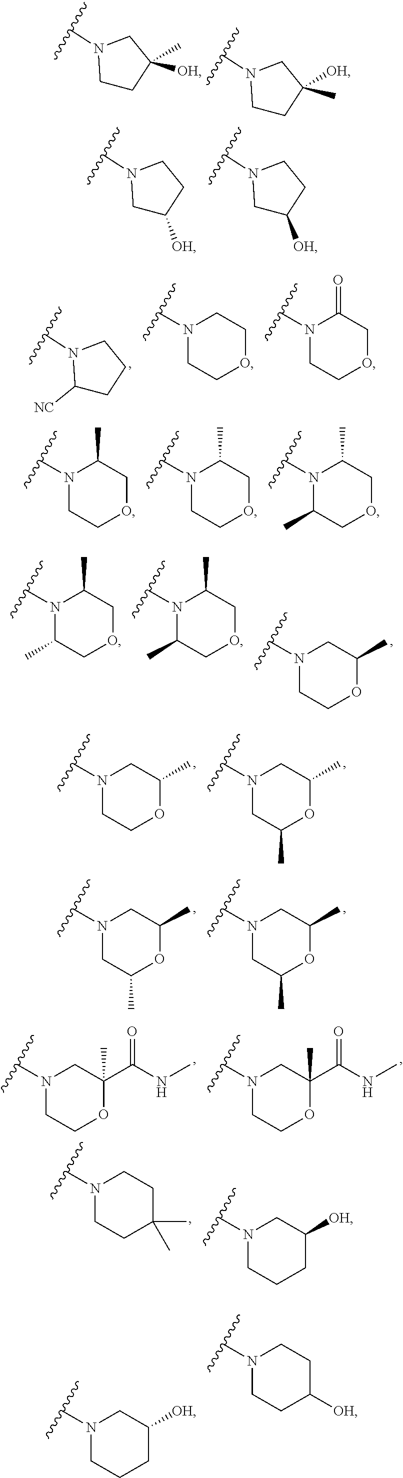

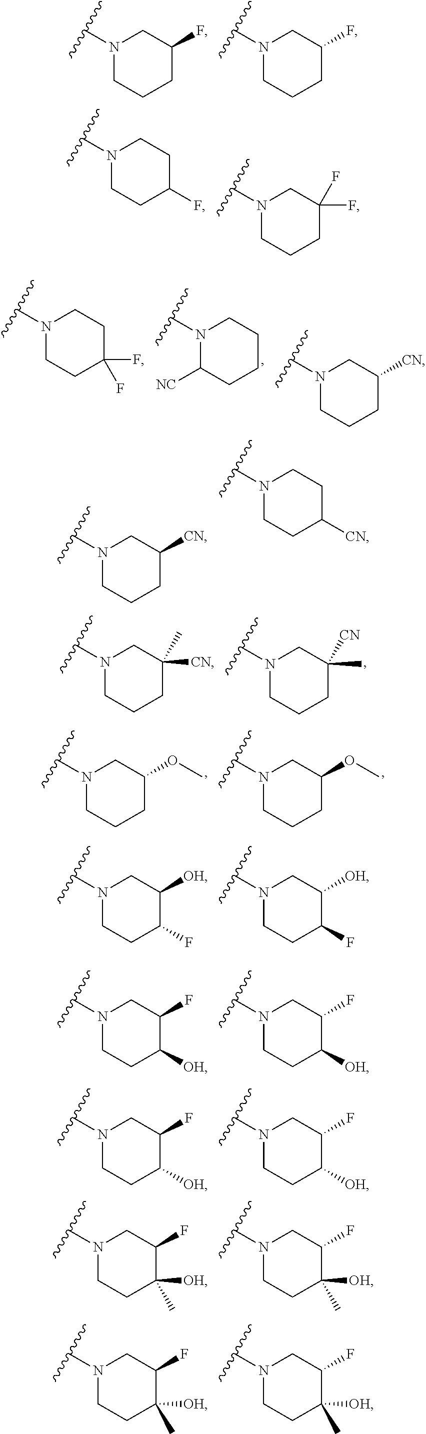

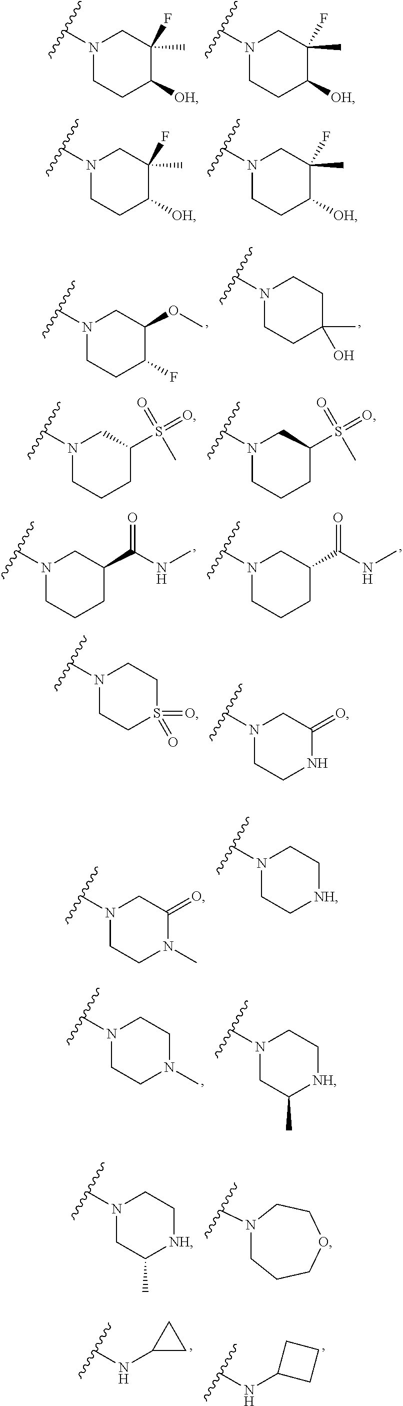

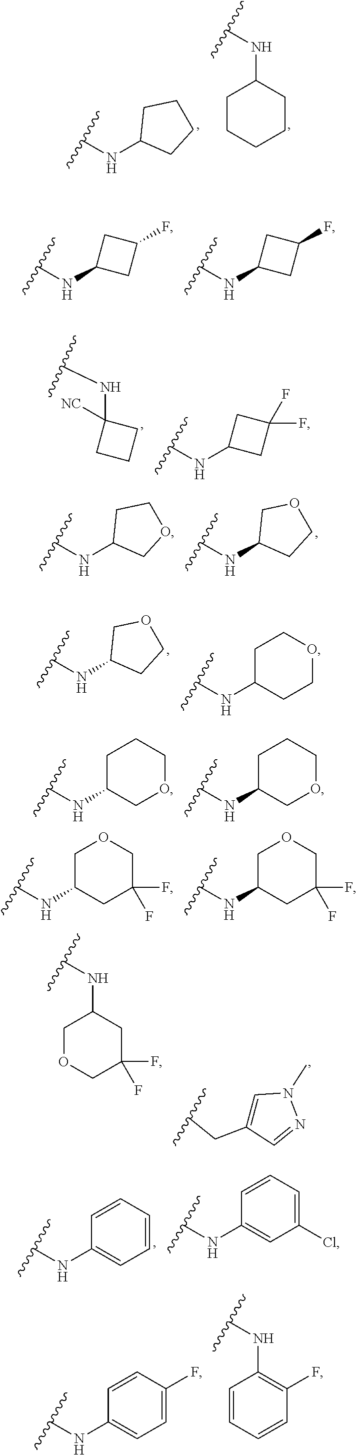

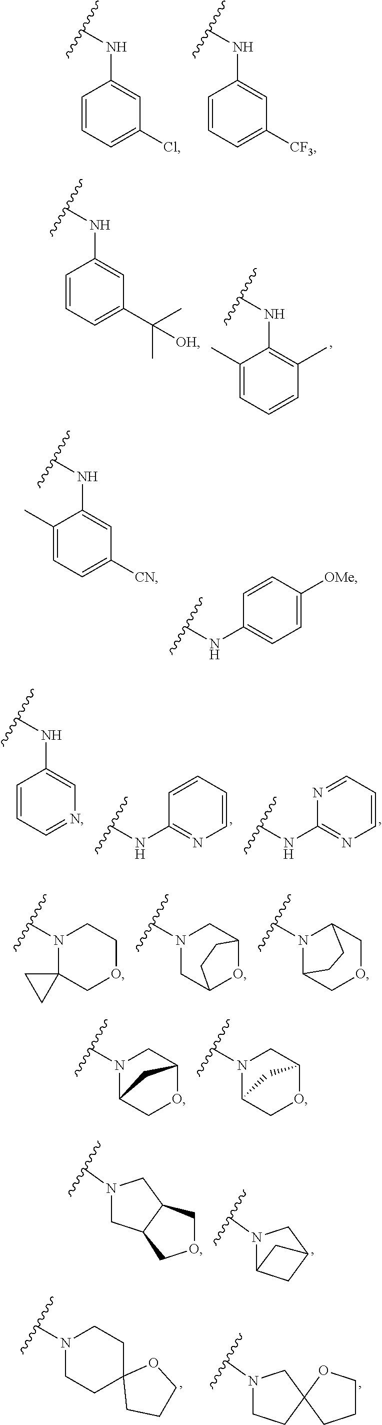

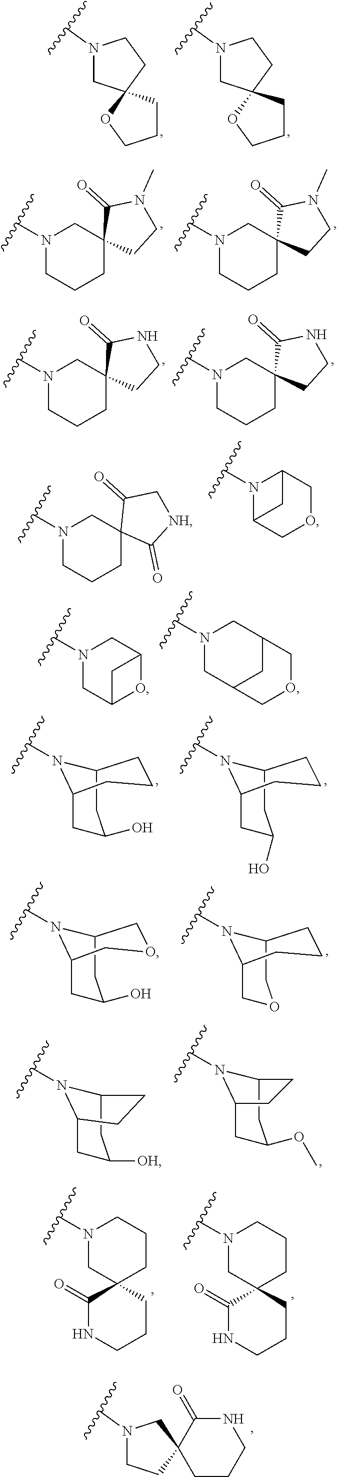

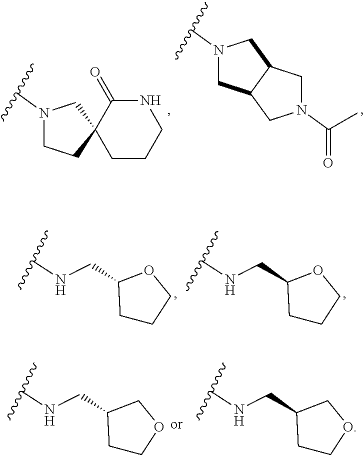

Aspect 12. The compound of anyone of the preceding Aspects, wherein R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{3c}; or
R^{3A} and R^{3B} together with the atom to which they are attached, form acyl (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is mono-ring, spiro ring, fused ring or bridged ring, said ring is optionally substituted with at least one substituent R³⁰;
R^{3c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, —C₁₋₈alkoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3d}, R^{3e} and R^{3f} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
preferably, R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; or
R^{3A} and R^{3B} together with the atom to which they are attached, form acyl (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is mono-ring, spiro ring, fused ring or bridged ring, said ring is optionally substituted with at least one substituent R^{3c};
R^{3c} is each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, oxo, —CN, —OR^{3d}, —COR^{3d}, —CO₂R^{3d}, —CONR^{3d}R^{3e}, —NO₂, —NR^{3d}R^{3e}, —NR^{3d}COR^{3e} or —SO₂R^{3d}, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3d} and R^{3e} are each independently selected from hydrogen, methyl, ethyl, propyl, butyl, pentyl, hexyl, heptyl, octyl, cyclopropyl, cyclobutyl, cyclopentyl, cyclohexyl, cycloheptyl or cyclooctyl, wherein each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent-F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
more preferably, R^{3A} and R^{3B} are each independently hydrogen, methyl, ethyl, propyl, butyl, pentyl; or
R^{3A} and R^{3B} together with the atom to which they are attached, form acyl (—C(═O)—) or a 3-, 4-, 5-, 6-, 7- or 8-membered ring, said ring comprising 0, 1 or 2 heteroatom(s) independently selected from nitrogen, oxygen or oxidized sulfur as ring member(s), said ring is mono-ring, spiro ring, fused ring or bridged ring, said ring is optionally substituted with at least one substituent selected from hydrogen, —F, methyl, ethyl, propyl, butyl, —CF₃, oxo or —CN.

Aspect 13. The compound of anyone of the preceding Aspects, wherein the

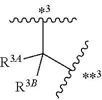

moiety is

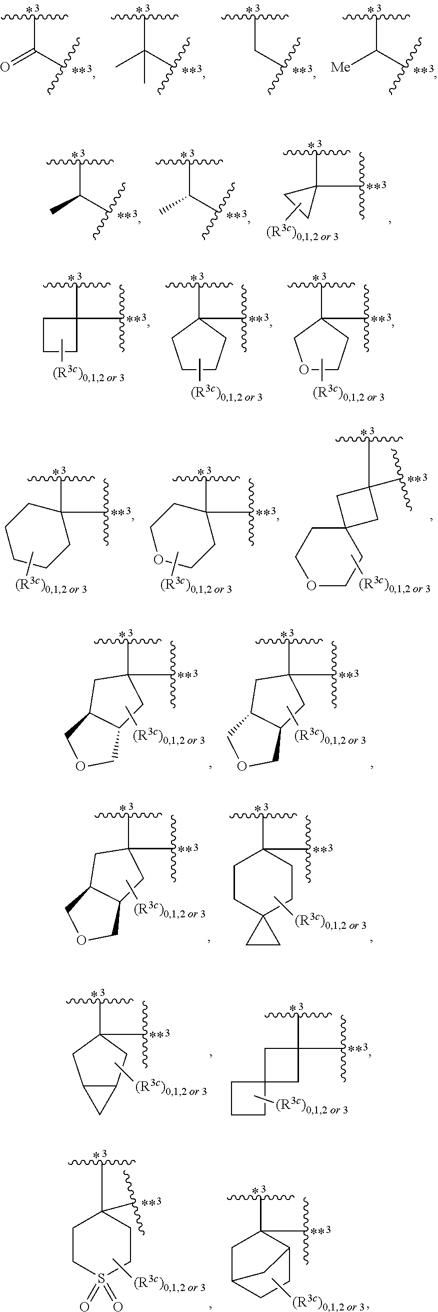

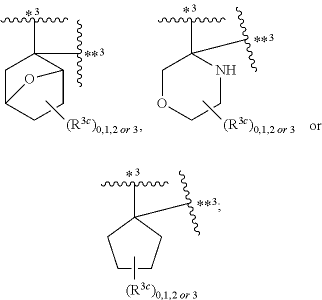

wherein *³ refers to the position attached to

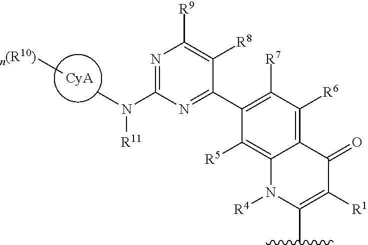

moiety, and **³ refers to the position attached to the

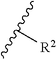

moiety;
preferably, the

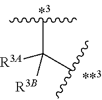

moiety is

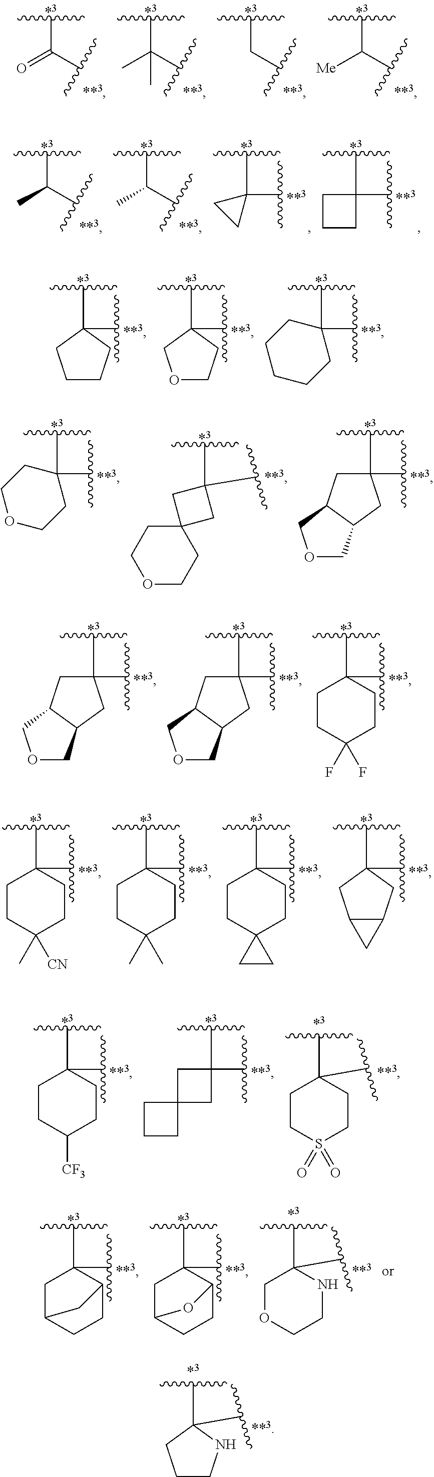

Aspect 14. The compound of anyone of the preceding Aspects, wherein the

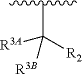

moiety is -Me, -Et,

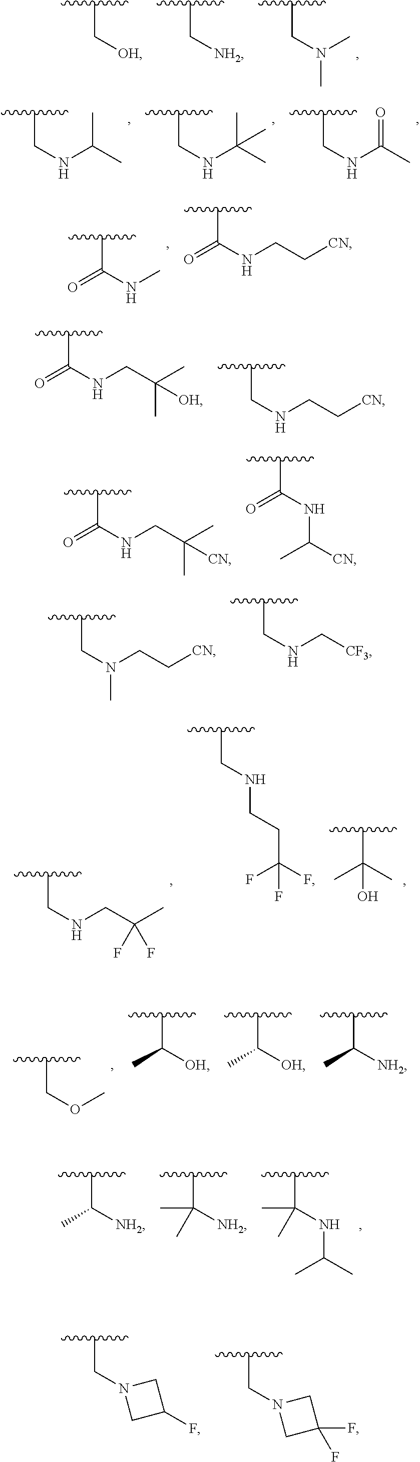

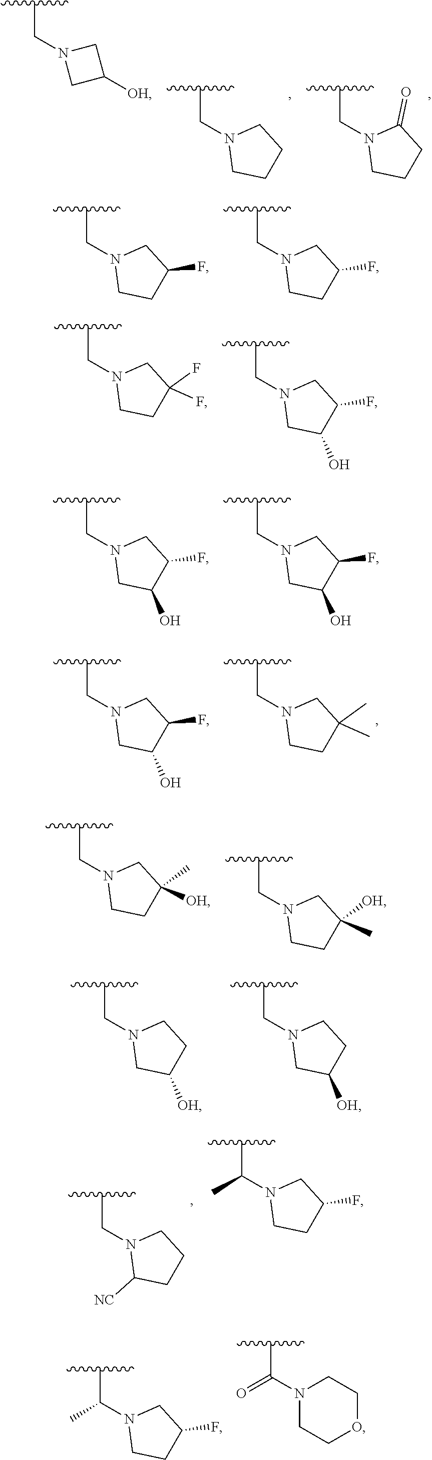

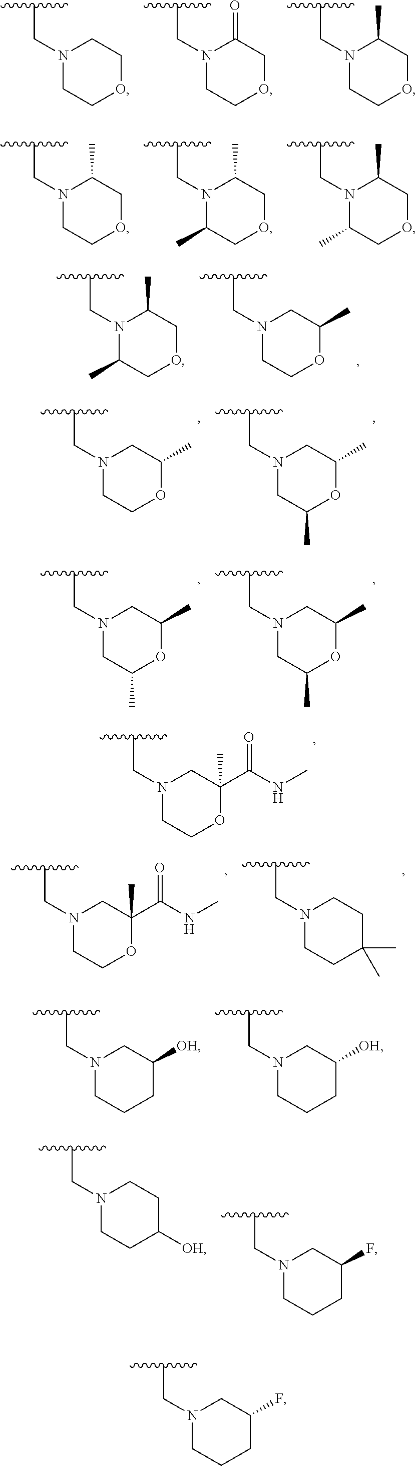

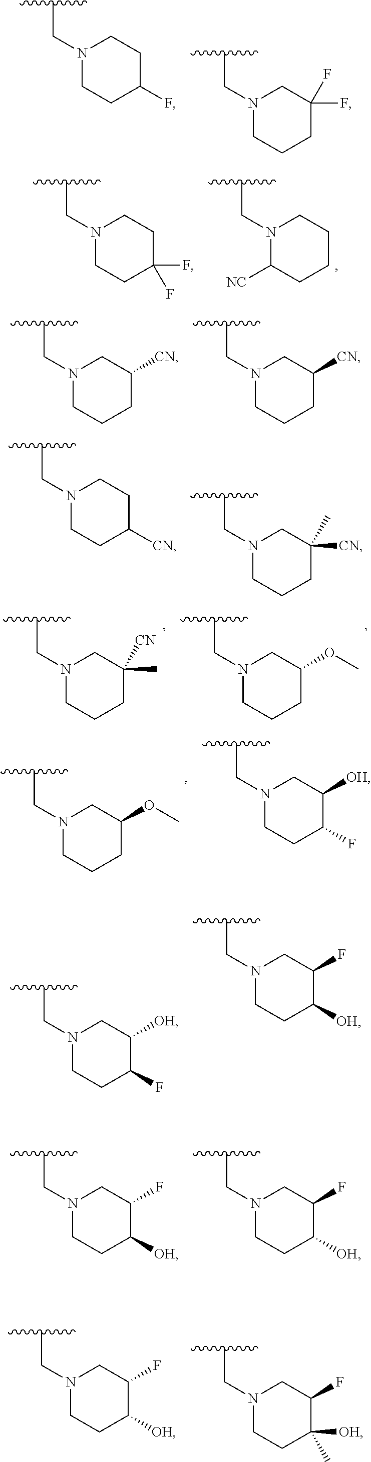

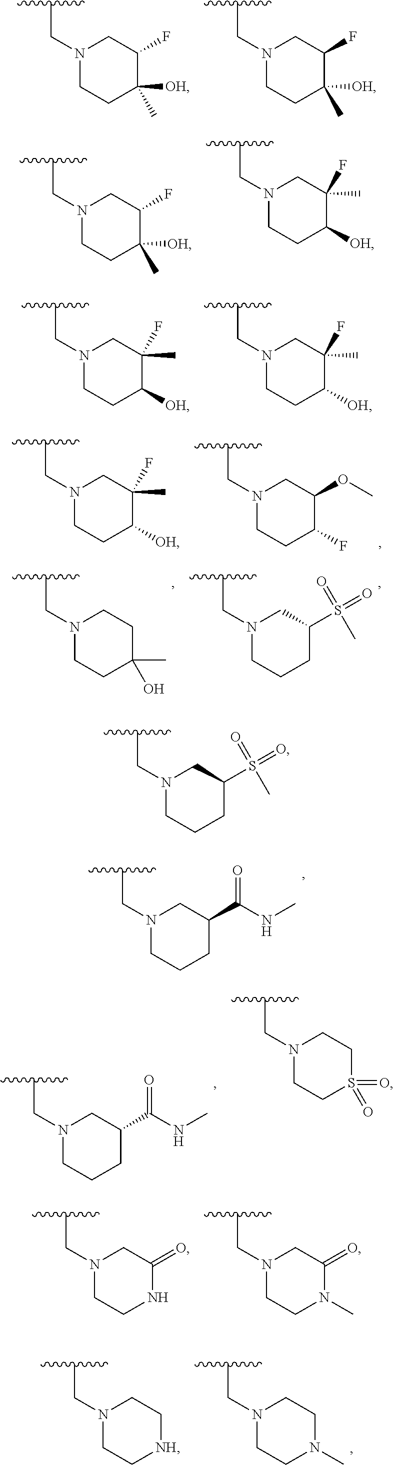

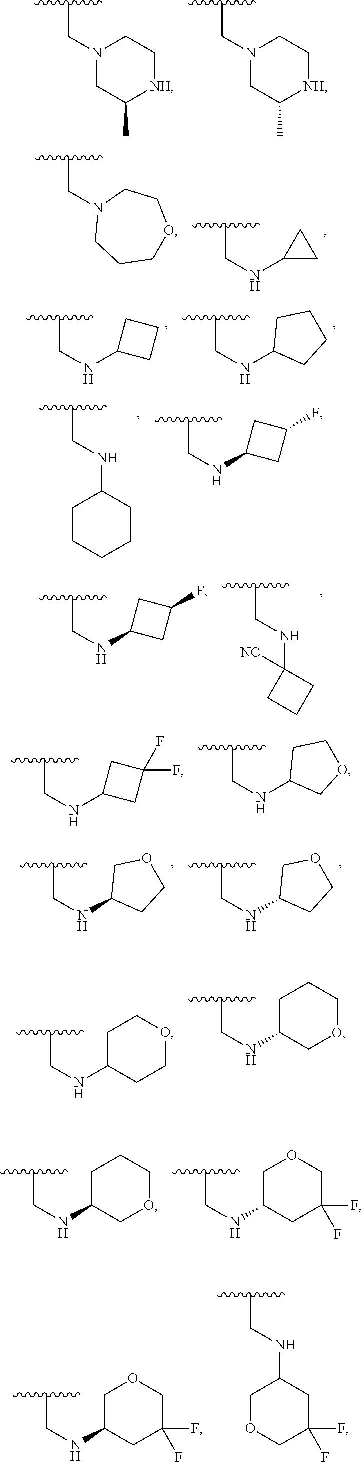

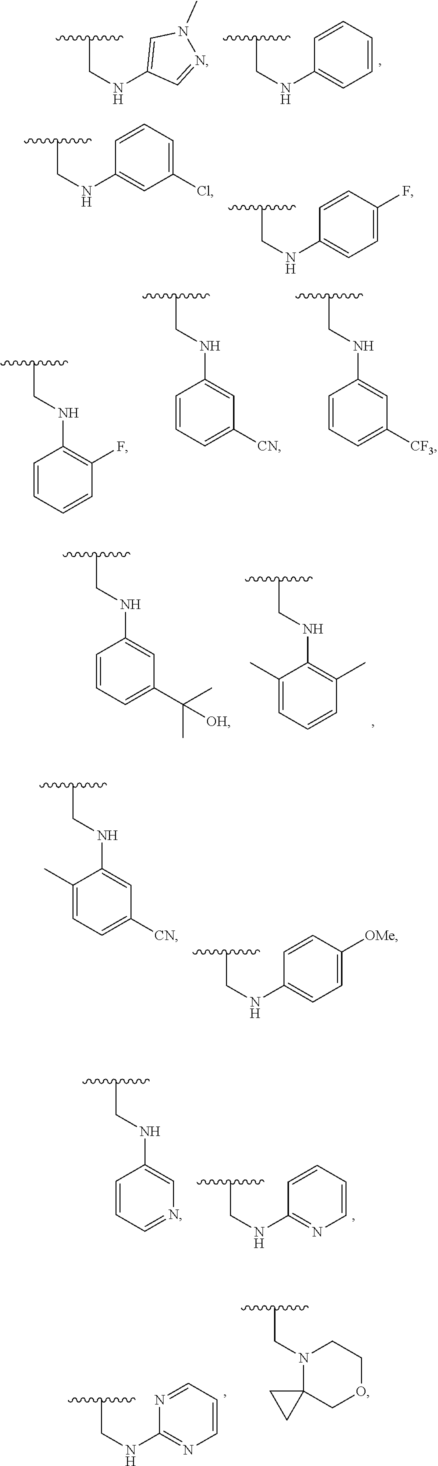

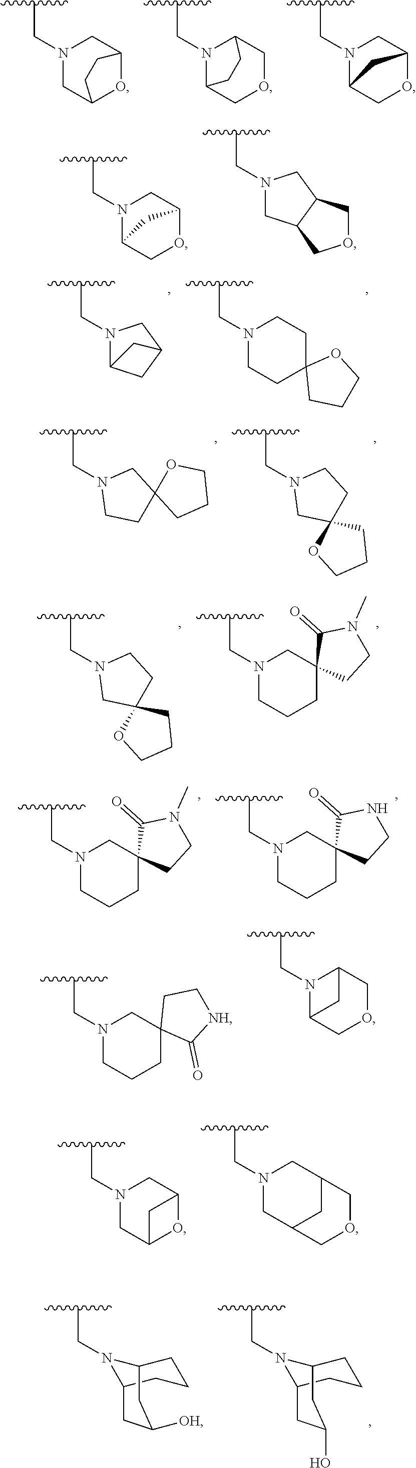

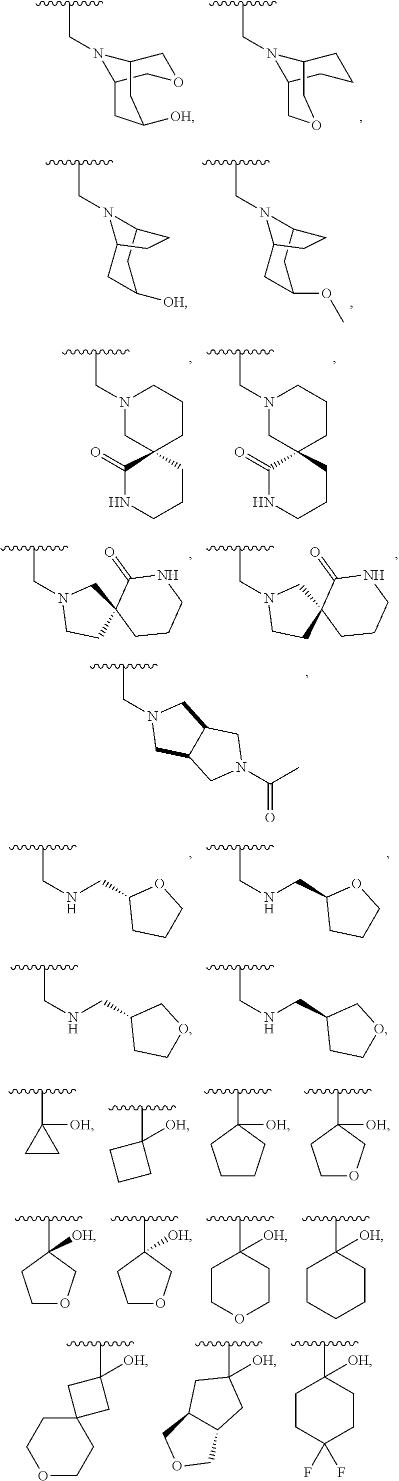

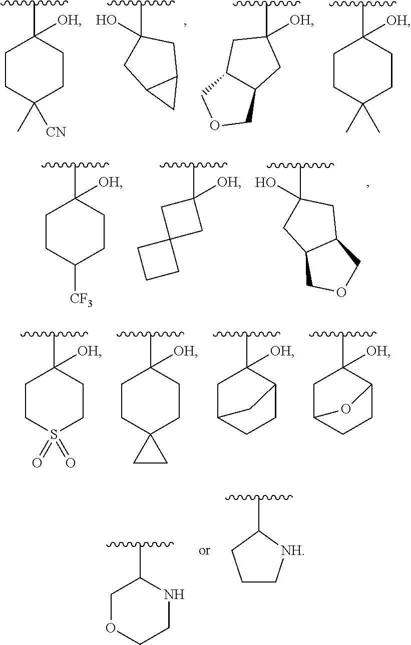

Aspect 15. The compound of anyone of the preceding Aspects, wherein R⁴ is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{401b}, —SO₂R^{401b}, —SO₂NR^{401b}R^{401c}, —COR^{401b}, —CO₂R^{401b}, —CONR^{401b}R^{401c}, —NO₂, —NR^{401b}R^{4c}, —NR^{401b}COR^{401b}, —NR^{401b}CO₂R^{401c}, —NR^{401b}CONR^{401c}R^{401d} or —NR^{401b}SO₂R^{401c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent R^{4a};
R^{401b}, R^{401c} and R^{401d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁴ is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, —COOMe, —COOEt, —COOC₃H₇, —COOCH(CH₃)₂, —COOCH₂CH₂CH₂CH₃, —COOCH(CH₃)CH₂CH₃, —COOCH₂CH(CH₃)₂, —COOC(CH₃) 3, —CH₂OH, —CH(CH₃)OH, —C(CH₃)₂OH, —CH₂NH₂, —CH(CH₃)NH₂ or —C(CH₃)₂NH₂;
more preferably, R⁴ is methyl, ethyl, n-propyl, iso-propyl, n-butyl, sec-butyl

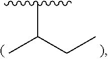 [SMILES: *C(C)CC]

iso-butyl

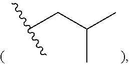 [SMILES: *CC(C)C]

tert-butyl

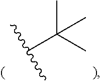 [SMILES: *C(C)(C)C]

—COOMe, —COOEt, —COOCH₂CH—CH₃, —COOCH(CH₃)₂, —COOCH₂CH₂CH₂CH₃, —COOCH(CH₃)CH—CH₃, —COOCH₂CH(CH₃)₂, —COOC(CH₃) 3, —CH₂OH, —CH(CH₃)OH, —C(CH₃)₂OH, —CH₂NH₂, —CH(CH₃) NH₂ or —C(CH₃) 2NH₂.

Aspect 16. The compound of anyone of the preceding Aspects, wherein R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R⁵⁶, —NR^{5a}R^{5b}, —NR^{5a}COR^{5b}, —NR^{5a}CO₂R^{5b} or —NR^{5a}CONR^{5b}R^{5c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5f};
R^{5d} and R^{5f} are each independently selected from hydrogen, —F, —Cl, —Br, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl;
more preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, methoxy, ethoxy, propoxy, butoxy, pentoxy, hexoxy, hepthoxy, octoxy, —CN.

Aspect 17. The compound of anyone of the preceding Aspects, wherein R⁵, R⁶ and R⁷ are each independently selected from H, —F, —Cl, —Br, —I, methyl, ethyl, propyl, butyl, pentyl, hexyl, heptyl, octyl; and/or
R⁸ is selected from —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, hepthoxy, octoxy, —CN; and/or
R⁹ is selected from H;
preferably, R⁵, R⁶ and R⁷ are each independently selected from H, —F, —Cl, methyl, ethyl, propyl, butyl; and/or
R⁸ is selected from —F, —Cl, methyl, ethyl, propyl, butyl, cyclopropyl, cyclobutyl, cyclopentyl, cyclohexyl, methoxy, ethoxy, propoxy, butoxy, —CN; and/or
R⁹ is selected from H.

Aspect 18. The compound of anyone of the preceding Aspects, wherein R¹¹ is selected from H, methyl, ethyl, propyl, butyl, pentyl, hexyl, heptyl, octyl, —C₂₋₈alkenyl or —C₂₋₈alkynyl; wherein each of said methyl, ethyl, propyl, butyl, pentyl, hexyl, heptyl, octyl, —C₂₋₈alkenyl or —C₂₋₈alkynyl is optionally substituted with at least one substituent R^{11a};
R^{11a} is selected from hydrogen, halogen, hydroxy, —C₁₋₈alkyl, -haloC₁₋₈alkyl, —C₁₋₈alkoxy, —C₂₋₈ alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; preferably, R¹¹ is selected from H, methyl, ethyl, propyl, butyl, pentyl, hexyl, heptyl, octyl, —C₂₋₈alkenyl or —C₂₋₈alkynyl;
more preferably, R¹¹ is selected from H, methyl, ethyl, propyl, butyl;
even more preferably, R¹¹ is H.

Aspect 19. The compound of anyone of the preceding Aspects, wherein m=0, the

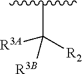

moiety as a whole is replaced with H.

Aspect 20. The compound of anyone of the preceding Aspects, wherein the compound is selected from

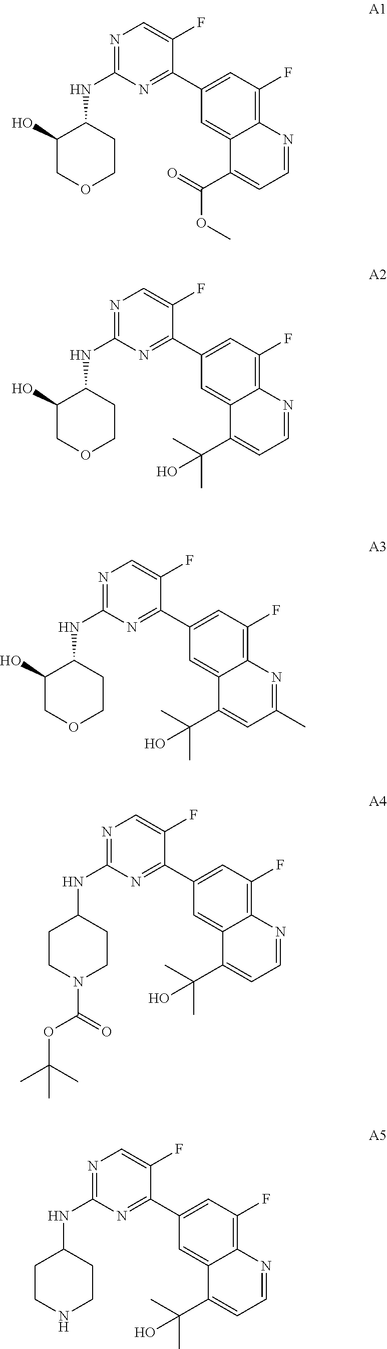

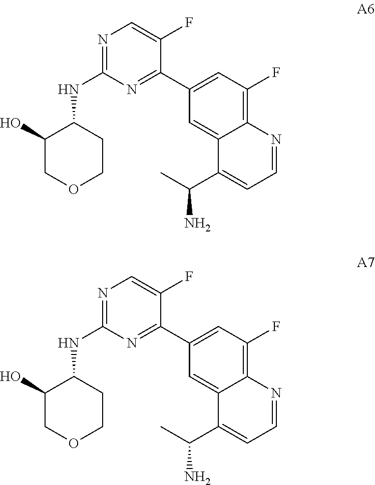 [SMILES: C[C@H](N)c1ccnc2c(F)cc(-c3nc(N[C@@H]4CCOC[C@H]4O)ncc3F)cc12 ; C[C@@H](N)c1ccnc2c(F)cc(-c3nc(N[C@@H]4CCOC[C@H]4O)ncc3F)cc12]

Aspect 21. A pharmaceutical composition comprising a compound of any one of Aspects 1-20 or a pharmaceutically acceptable salt, stereoisomer, tautomer or prodrug thereof, together with a pharmaceutically acceptable excipient.

Aspect 22. A method of decreasing CDK4 activity by inhibition, which comprises administering to an individual the compound according to any one of Aspects 1-19, or a pharmaceutically acceptable salt thereof, including the compound of formula (I) or the specific compounds exemplified herein.

Aspect 23. The method of Aspect 22, wherein the disease is selected from cancer, preferred breast cancer, lung cancer, pancreatic cancer, prostate cancer, bone cancer, liver cancer and endometrial cancer.

Aspect 24. Use of a compound of any one of Aspects 1-20 or a pharmaceutically acceptable salt, stereoisomer, tautomer or prodrug thereof in the preparation of a medicament for treating a disease that can be affected by CDK4 modulation.

Aspect 25. The use of Aspect 24, wherein the disease is cancer, preferred breast cancer, lung cancer, pancreatic cancer, prostate cancer, bone cancer, liver cancer and endometrial cancer.

Aspect 26. A method of treating a disease or disorder in a patient comprising administering to the patient a therapeutically effective amount of the compound any one of Aspects 1-20, or a pharmaceutically acceptable salt thereof as a CDK4 kinase inhibitor, wherein the disease or disorder is associated with inhibition of CDK4.

Aspect 27. The method of Aspect 26, wherein the disease is selected from cancer, preferred breast cancer, lung cancer, pancreatic cancer, prostate cancer, bone cancer, liver cancer and endometrial cancer.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C≡C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈ cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur(S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group, the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113 (3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl-CH₂C(O)— groups (keto forms) may undergo tautomerism to form hydroxyl-CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### General Synthesis

Compounds disclosed herein, including salts thereof, can be prepared using known organic synthesis techniques and can be synthesized according to any of numerous possible synthetic routes. The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the boiling temperature of solvent. A given reaction can be carried out in one solvent or mixture of solvents.

The selection of appropriate protecting group, can be readily determined by one skilled in the art. Reactions can be monitored according to any suitable method known in the art, such as NMR, UV, HPLC, LC-MS and TLC. Compounds can be purified by a variety of methods, including HPLC and normal phase silica chromatography.

Chiral analytic HPLC was used for the retention time analysis of different chiral examples, the conditions were divided into the methods as below according to the column, mobile phase, solvent ration used.

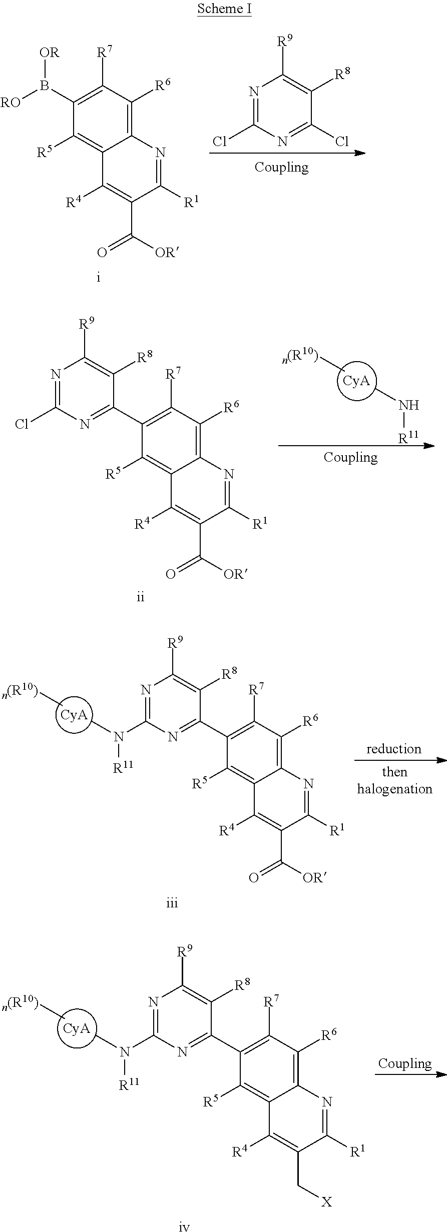

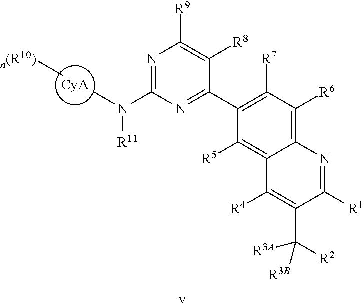

For example, compounds of Formulas (I), (II), (III), or (IV) can be formed as shown in Scheme I. The compound (i) can react with halogenated pyrimidine under palladium catalyzed reaction condition or base mediated coupling condition to give compound (ii) that can couple with amine to give compound (iii), reduction and halogenation of compound (iii) give compound (iv) which can be used for coupling to give compound (v).

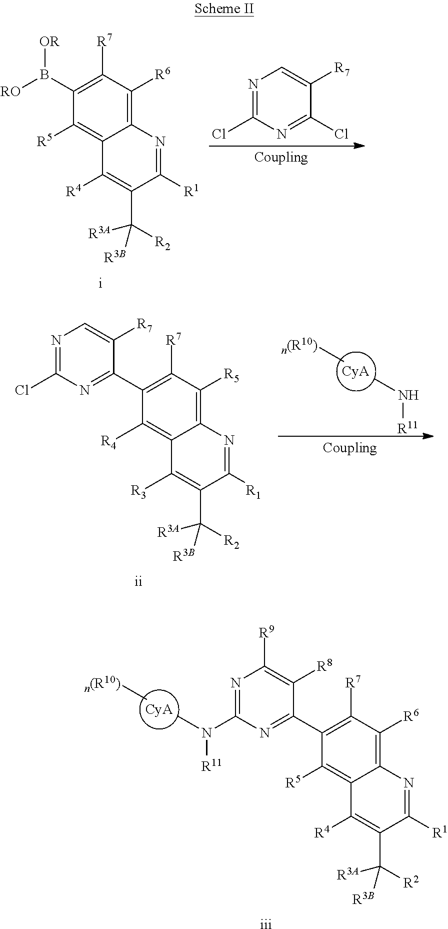

For example, compounds of Formulas (I), (II), (AIII), (III), (AIV) or (IV) can be formed as shown in Scheme II. The compound (i) can react with halogenated pyrimidine under palladium catalyzed reaction condition to give compound (ii) that can couple with amine to give compound (iii).

### EXAMPLES

### Example A1: methyl 8-fluoro-6-(5-fluoro-2-(((3S,4R)-3-hydroxytetrahydro-2H-pyran-4-yl)amino)pyrimidin-4-yl) quinoline-4-carboxylate

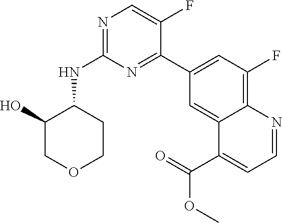 [SMILES: COC(=O)c1ccnc2c(F)cc(-c3nc(N[C@@H]4CCOC[C@H]4O)ncc3F)cc12]

### Step 1:5-(((4-chloro-2-fluorophenyl)amino)methylene)-2,2-dimethyl-1,3-dioxane-4,6-dione

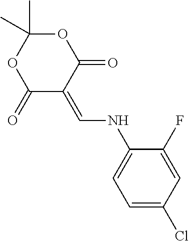 [SMILES: CC1(C)OC(=O)C(=CNc2ccc(Cl)cc2F)C(=O)O1]

A mixture of 4-chloro-2-fluoroaniline (50 g, 343.50 mmol) and 5-(methoxymethylidene)-2,2-dimethyl-1,3-dioxane-4,6-dione (65.0 g, 349.16 mmol) in isopropanol (500 mL) was stirred for 4 h at 85 °C under nitrogen atmosphere before cooled to room temperature. The precipitated solid was collected by filtration and washed with isopropanol (2×20 mL) to yield the title compound (95 g, 92%). LC-MS (M+H)⁺=299.9.

### Step 2:6-chloro-8-fluoroquinolin-4-ol

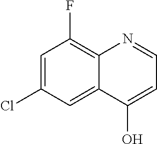 [SMILES: Oc1ccnc2c(F)cc(Cl)cc12]

A mixture of 5-(((4-chloro-2-fluorophenyl)amino)methylene)-2,2-dimethyl-1,3-dioxane-4,6-dione (36 g, 120.128 mmol) in phenoxybenzene (240 mL) was stirred for 1.5 h at 265 °C under nitrogen atmosphere before cooled to room temperature. The resulting mixture was diluted with petroleum ether (240 mL) and filtered. The precipitated solid was washed with petroleum ether (2×20 mL). The residue was diluted with ethyl acetate (60 mL). The precipitated solids were collected by filtration and washed with ethyl acetate (1×10 mL) to yield the title compound (6.1 g, 25%). LC-MS (M+H)⁺=197.9.

### Step 3:4-bromo-6-chloro-8-fluoroquinoline

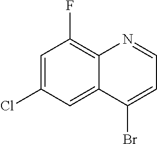 [SMILES: Fc1cc(Cl)cc2c(Br)ccnc12]

To a solution of 6-chloro-8-fluoroquinolin-4-ol (8.1 g, 40.99 mmol) in N,N-dimethylformamide (130 mL) was added phosphorus tribromide (22.8 g, 84.23 mmol) dropwise at 0 °C under nitrogen atmosphere. The resulting mixture was stirred for 16 h at room temperature under nitrogen atmosphere before quenched by addition of water (200 mL). The mixture was basified to pH 8 with saturated sodium bicarbonate solution. The precipitated solids were collected by filtration and washed with water (2×25 mL). The crude solid was purified by flash chromatography eluting with ethyl acetate in petroleum ether (0-15% gradient, v/v) to yield the title compound (6 g, 56%). LC-MS (M+H)⁺=259.8.

### Step 4: methyl 6-chloro-8-fluoroquinoline-4-carboxylate

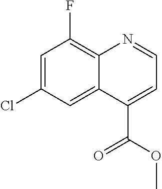 [SMILES: COC(=O)c1ccnc2c(F)cc(Cl)cc12]

To a solution of 4-bromo-6-chloro-8-fluoroquinoline (200 mg, 0.768 mmol) in 8 mL methanol was added triethylamine (234 mg, 2.304 mmol), (1,1′-bis(diphenylphosphino) ferrocene)dichloropalladium-dichloromethane (1:1) (188 mg, 0.23 mmol) in a pressure tank. The mixture was pressurized to 40 atm with carbon monoxide at 80 °C for 12 h before cooled to room temperature. The resulting mixture was concentrated under reduced pressure and the residue was purified by flash chromatography eluting with ethyl acetate in petroleum ether (0-20% gradient, v/v) to yield the title compound (150 mg, 87%). LC-MS (M+H)⁺=239.9.

### Step 5: (8-fluoro-4-(methoxycarbonyl) quinolin-6-yl) boronic acid

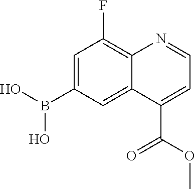 [SMILES: COC(=O)c1ccnc2c(F)cc(B(O)O)cc12]

To a solution of methyl 6-chloro-8-fluoroquinoline-4-carboxylate (90 mg, 0.376 mmol) and bis(pinacolato)diboron (190 mg, 0.748 mmol) in 1,4-dioxane (3 mL) were added potassium acetate (110 mg, 1.12 mmol) and dichlorobis(tricyclohexylphosphine) palladium (II) (35 mg, 0.047 mmol). The resulting mixture was stirred for 3 h at 100 °C under nitrogen atmosphere before cooled to room temperature. The resulting mixture was concentrated under reduced pressure and diluted with ethyl acetate (10 mL). The resulting mixture was filtered, and the filter cake was washed with ethyl acetate (2×5 mL). The filtrate was combined and concentrated under reduced pressure to yield the title compound (90 mg, crude). The crude product was used in the next step directly without further purification. LC-MS (M+H)⁺=250.1.

### Step 6: methyl 6-(2-chloro-5-fluoropyrimidin-4-yl)-8-fluoroquinoline-4-carboxylate

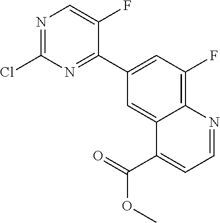 [SMILES: COC(=O)c1ccnc2c(F)cc(-c3nc(Cl)ncc3F)cc12]

To a solution of (8-fluoro-4-(methoxycarbonyl) quinolin-6-yl) boronic acid (90 mg, crude) and 2,4-dichloro-5-fluoropyrimidine (50 mg, 0.299 mmol) in 1,4-dioxane (6 mL) and water (0.6 mL) were added potassium phosphate (120 mg, 0.565 mmol) and (1,1′-bis(diphenylphosphino) ferrocene) palladium (II) dichloride (25 mg, 0.034 mmol). After stirring for 3 h at 100 °C under nitrogen atmosphere, the resulting mixture was cooled to room temperature and concentrated under reduced pressure. The residue was purified by flash chromatography eluting with ethyl acetate in petroleum ether (0-40% gradient, v/v) to yield the title compound (67 mg, 51% for 2 steps). LC-MS (M+H)⁺=335.9.

### Step 7: methyl 8-fluoro-6-(5-fluoro-2-(((3S,4R)-3-hydroxytetrahydro-2H-pyran-4-yl)amino)pyrimidin-4-yl) quinoline-4-carboxylate

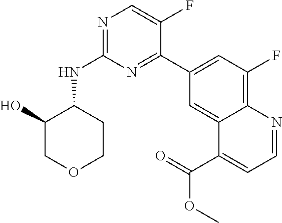 [SMILES: COC(=O)c1ccnc2c(F)cc(-c3nc(N[C@@H]4CCOC[C@H]4O)ncc3F)cc12]

A mixture of methyl 6-(2-chloro-5-fluoropyrimidin-4-yl)-8-fluoroquinoline-4-carboxylate (60 mg, 0.179 mmol), (SP-4-1)-[1,3-bis[2,6-bis(1-ethylpropyl)phenyl]-4,5-dichloro-1,3-dihydro-2H-imidazol-2-ylidene]dichloro(2-methylpyridine) palladium (15 mg, 0.018 mmol), cesium carbonate (174 mg, 0.537 mmol) and (3S,4R)-4-aminooxan-3-ol hydrochloride (55 mg, 0.358 mmol) in 1,4-dioxane (3 mL) was stirred for 3 h at 100 °C under nitrogen atmosphere before cooled to room temperature. The resulting mixture was concentrated under reduced pressure and the residue was purified by prep-HPLC under the following conditions: column, XBridge Shield RP18 OBD Column, 30×150 mm, 5 μm; mobile phase, acetonitrile in water (with 10 mmol/L ammonium bicarbonate and 0.1% ammonium hydroxide), 23% to 53% gradient in 8 min; detector, UV 254 nm. The title compound (22 mg, 29%) obtained. ¹H NMR (300 MHz, DMSO-d6) δ 9.25 (s, 1H), 9.20 (d, J=3.8 Hz, 1H), 8.53 (d, J=3.8 Hz, 1H), 8.27-8.17 (m, 1H), 8.13 (d, J=4.4 Hz, 1H), 7.35 (d, J=7.8 Hz, 1H), 4.97 (d, J=4.8 Hz, 1H), 4.02 (s, 3H), 3.91-3.80 (m, 3H), 3.61-3.35 (m, 2H), 3.10 (t, J=10.3 Hz, 1H), 2.11-2.00 (m, 1H), 1.61-1.42 (m, 1H). LC-MS (M+H)⁺=417.0.

### Example A2: (3S,4R)-4-((5-fluoro-4-(8-fluoro-4-(2-hydroxypropan-2-yl) quinolin-6-yl)pyrimidin-2-yl)amino)tetrahydro-2H-pyran-3-ol

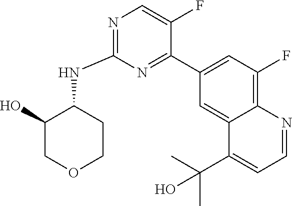 [SMILES: CC(C)(O)c1ccnc2c(F)cc(-c3nc(N[C@@H]4CCOC[C@H]4O)ncc3F)cc12]

### Step 1:2-(6-chloro-8-fluoroquinolin-4-yl) propan-2-ol

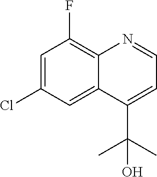 [SMILES: CC(C)(O)c1ccnc2c(F)cc(Cl)cc12]

To a mixture of methyl 6-chloro-8-fluoroquinoline-4-carboxylate (750 mg, 3.13 mmol) in tetrahydrofuran (20 mL) were added methylmagnesium iodide (1M in diethyl ether, 15.6 mL, 15.6 mmol) dropwise at 0 °C under nitrogen atmosphere. The resulting mixture was stirred for 4 h at 0 °C under nitrogen atmosphere before addition of saturated ammonium chloride solution (20 mL) at 0 °C. The resulting solution was extracted with ethyl acetate (50 mL×3). The organic phases were combined, washed with brine, dried over sodium sulfate, filtered and concentrated. The residue was purified by flash chromatography eluting with ethyl acetate in petroleum ether (0-50% gradient, v/v) to yield the title compound (425 mg, 56%). LC-MS (M+H)⁺=240.0.

### Step 2: (8-fluoro-4-(2-hydroxypropan-2-yl) quinolin-6-yl) boronic acid

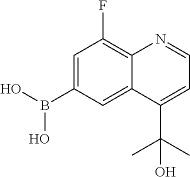 [SMILES: CC(C)(O)c1ccnc2c(F)cc(B(O)O)cc12]

The title compound (375 mg, crude) was prepared in a manner similar to that in Example A1 step 5 from 2-(6-chloro-8-fluoroquinolin-4-yl) propan-2-ol. The crude product was used in the next step directly without further purification. LC-MS (M+H)⁺=250.0.

### Step 3:2-(6-(2-chloro-5-fluoropyrimidin-4-yl)-8-fluoroquinolin-4-yl) propan-2-ol

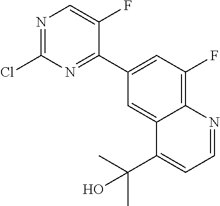 [SMILES: CC(C)(O)c1ccnc2c(F)cc(-c3nc(Cl)ncc3F)cc12]

The title compound (400 mg, 67%, 2 steps) was prepared in a manner similar to that in Example A1 step 6 from (8-fluoro-4-(2-hydroxypropan-2-yl) quinolin-6-yl) boronic acid and 2,4-dichloro-5-fluoropyrimidine. LC-MS (M+H)⁺=335.9.

### Step 4: (3S,4R)-4-((5-fluoro-4-(8-fluoro-4-(2-hydroxypropan-2-yl) quinolin-6-yl)pyrimidin-2-yl)amino)tetrahydro-2H-pyran-3-ol

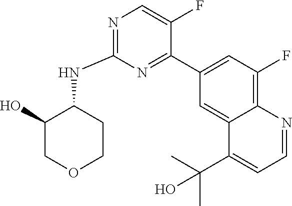 [SMILES: CC(C)(O)c1ccnc2c(F)cc(-c3nc(N[C@@H]4CCOC[C@H]4O)ncc3F)cc12]

The title compound (11 mg, 11%) was prepared in a manner similar to that in Example A1 step 7 from 2-(6-(2-chloro-5-fluoropyrimidin-4-yl)-8-fluoroquinolin-4-yl) propan-2-ol and (3S,4R)-4-aminooxan-3-ol hydrochloride. ¹H NMR (300 MHz, DMSO-d6) δ 9.46 (s, 1H), 8.96 (d, J=4.6 Hz, 1H), 8.51 (d, J=3.8 Hz, 1H), 8.12-8.02 (m, 1H), 7.72 (d, J=4.6 Hz, 1H), 7.30 (d, J=7.7 Hz, 1H), 5.73 (s, 1H), 4.96 (d, J=5.3 Hz, 1H), 3.88-3.77 (m, 3H), 3.58-3.35 (m, 2H), 3.07 (t, J=10.4 Hz, 1H), 2.08-1.99 (m, 1H), 1.74 (s, 6H), 1.54-1.43 (m, 1H). LC-MS (M+H)⁺=417.2.

### Example A3: (3S,4R)-4-((5-fluoro-4-(8-fluoro-4-(2-hydroxypropan-2-yl)-2-methylquinolin-6-yl)pyrimidin-2-yl)amino)tetrahydro-2H-pyran-3-ol

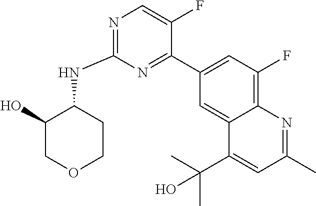 [SMILES: Cc1cc(C(C)(C)O)c2cc(-c3nc(N[C@@H]4CCOC[C@H]4O)ncc3F)cc(F)c2n1]

### Step 1:5-chloro-7-fluoroindoline-2,3-dione

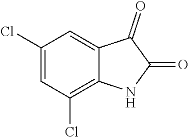 [SMILES: O=C1Nc2c(Cl)cc(Cl)cc2C1=O]

To a solution of 7-fluoro-1H-indole-2,3-dione (950 mg, 5.753 mmol) in sulfuric acid (5 mL) and trifluoroacetic acid (5 mL) was added trichloro-1,3,5-triazinane-2,4,6-trione (665 mg, 2.861 mmol) in portions at 0 °C under nitrogen atmosphere. Then the resulting mixture was stirred for 4 h at room temperature under nitrogen atmosphere before quenched by the addition of water (25 mL) at 0 °C. The precipitated solids were collected by filtration and washed with water (2×5 mL) to yield the title compound (800 mg, 69%). LC-MS (M+H)⁺=200.1.

### Step 2:6-chloro-8-fluoro-2-methylquinoline-4-carboxylic acid

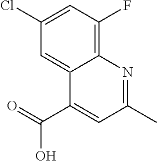 [SMILES: Cc1cc(C(=O)O)c2cc(Cl)cc(F)c2n1]

To a stirred solution of 5-chloro-7-fluoro-1H-indole-2,3-dione (600 mg, 3.00 mmol) and acetone (620 μL, 8.40 mmol) in ethanol (3.5 mL) and water (1.2 mL) was added potassium hydroxide (472 mg, 8.40 mmol) in portions at room temperature under nitrogen atmosphere. The reaction mixture was irradiated with microwave for 20 min at 100 °C. The resulting mixture was concentrated under reduced pressure and then diluted with water (15 mL). The resulting mixture was washed with dichloromethane (20 mL). The aqueous layer was acidified to pH 3 with 2 N HCl solution and then extracted with ethyl acetate (2×15 mL). The organic phases were combined, washed with brine, dried over sodium sulfate, filtered and concentrated. The crude product (580 mg) was used in the next step directly without further purification. LC-MS (M+H)⁺=239.9.

### Step 3: methyl 6-chloro-8-fluoro-2-methylquinoline-4-carboxylate

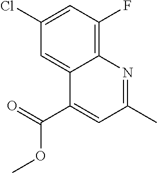 [SMILES: COC(=O)c1cc(C)nc2c(F)cc(Cl)cc12]

To a solution of 6-chloro-8-fluoro-2-methylquinoline-4-carboxylic acid (580 mg, 2.835 mmol) in methanol (30 mL) was added sulfuric acid (0.2 mL) dropwise at room temperature under nitrogen atmosphere. The resulting mixture was stirred for 16 h at 100 °C under nitrogen atmosphere before cooled to room temperature and concentrated under reduced pressure. The resulting mixture was diluted with water (15 mL) and extracted with ethyl acetate (2×15 mL). The organic phases were combined, washed with brine, dried over sodium sulfate, filtered and concentrated. The residue was purified by flash chromatography eluting with ethyl acetate in petroleum ether (0% to 30% gradient, v/v) to yield the title compound (184 mg, 24% yield for 2 steps). LC-MS (M+H)⁺=253.9.

### Step 4:2-(6-chloro-8-fluoro-2-methylquinolin-4-yl) propan-2-ol

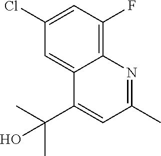 [SMILES: Cc1cc(C(C)(C)O)c2cc(Cl)cc(F)c2n1]

The title compound (153 mg, 83%) was prepared in a manner similar to that in Example A2 step 1 from methyl 6-chloro-8-fluoro-2-methylquinoline-4-carboxylate. LC-MS (M+H)⁺=254.0.

### Step 5: (8-fluoro-4-(2-hydroxypropan-2-yl)-2-methylquinolin-6-yl) boronic acid

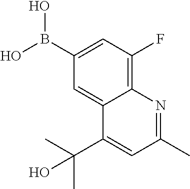 [SMILES: Cc1cc(C(C)(C)O)c2cc(B(O)O)cc(F)c2n1]

The title compound (70 mg, crude) was prepared in a manner similar to that in Example A1 step 5 from 2-(6-chloro-8-fluoro-2-methylquinolin-4-yl) propan-2-ol. The crude product was used in the next step directly without further purification. LC-MS (M+H)⁺=264.0.

### Step 6:2-(6-(2-chloro-5-fluoropyrimidin-4-yl)-8-fluoro-2-methylquinolin-4-yl) propan-2-ol

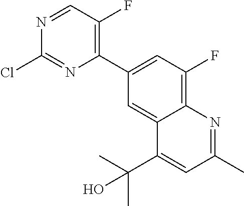 [SMILES: Cc1cc(C(C)(C)O)c2cc(-c3nc(Cl)ncc3F)cc(F)c2n1]

The title compound (10 mg, 9%, 2 steps) was prepared in a manner similar to that in Example A1 step 6 from (8-fluoro-4-(2-hydroxypropan-2-yl)-2-methylquinolin-6-yl) boronic acid and 2,4-dichloro-5-fluoropyrimidine. LC-MS (M+H)⁺=350.0.

### Step 7: (3S,4R)-4-((5-fluoro-4-(8-fluoro-4-(2-hydroxypropan-2-yl)-2-methylquinolin-6-yl)pyrimidin-2-yl)amino)tetrahydro-2H-pyran-3-ol

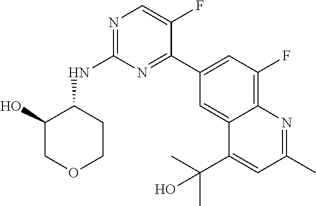 [SMILES: Cc1cc(C(C)(C)O)c2cc(-c3nc(N[C@@H]4CCOC[C@H]4O)ncc3F)cc(F)c2n1]

The title compound (4 mg, 38%) was prepared in a manner similar to that in Example A1 step 7 from 2-(6-(2-chloro-5-fluoropyrimidin-4-yl)-8-fluoro-2-methylquinolin-4-yl) propan-2-ol and (3S,4R)-4-aminooxan-3-ol hydrochloride. ¹H NMR (300 MHz, DMSO-d6) δ 9.39 (s, 1H), 8.49 (d, J=3.9 Hz, 1H), 8.09-7.99 (m, 1H), 7.61 (s, 1H), 7.25 (d, J=7.6 Hz, 1H), 5.67 (s, 1H), 4.97 (d, J=5.3 Hz, 1H), 3.89-3.79 (m, 3H), 3.57-3.40 (m, 2H), 3.14-3.01 (m, 1H), 2.72 (s, 3H), 2.12-1.95 (m, 1H), 1.73 (s, 6H), 1.58-1.42 (m, 1H). LC-MS (M+H)⁺=431.1

### Example A4: tert-butyl 4-((5-fluoro-4-(8-fluoro-4-(2-hydroxypropan-2-yl) quinolin-6-yl)pyrimidin-2-yl)amino) piperidine-1-carboxylate

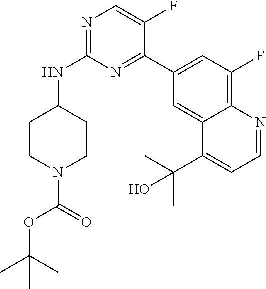 [SMILES: CC(C)(C)OC(=O)N1CCC(Nc2ncc(F)c(-c3cc(F)c4nccc(C(C)(C)O)c4c3)n2)CC1]

A mixture of 2-(6-(2-chloro-5-fluoropyrimidin-4-yl)-8-fluoroquinolin-4-yl) propan-2-ol (200 mg, 0.6 mmol), (SP-4-1)-[1,3-bis[2,6-bis(1-ethylpropyl)phenyl]-4,5-dichloro-1,3-dihydro-2H-imidazol-2-ylidene]dichloro(2-methylpyridine) palladium (51 mg, 0.059 mmol), cesium carbonate (582, 1.79 mmol) and tert-butyl 4-aminopiperidine-1-carboxylate (239 mg, 1.19 mmol) in 1,4-dioxane (15 mL) was stirred for 3 h at 100 °C under nitrogen atmosphere before cooled to room temperature. The resulting mixture was concentrated under reduced pressure. The residue was purified silica gel column chromatography eluting with 16% ethyl acetate in petroleum ether (v/v) and repurified by prep-HPLC under the following conditions: column, XBridge Shield RP18 OBD Column, 30×150 mm, 5 μm; mobile phase, acetonitrile in water (with 10 mmol/L ammonium bicarbonate and 0.05% ammonium hydroxide), 25% to 55% gradient in 8 min; detector, UV 254 nm. The title compound (110 mg, 41%) was obtained as a white solid. ¹H NMR (300 MHz, DMSO-d6) δ 9.46 (s, 1H), 8.96 (d, J=4.6 Hz, 1H), 8.52 (d, J=3.8 Hz, 1H), 8.12-8.02 (m, 1H), 7.71 (d, J=4.7 Hz, 1H), 7.39 (d, J=7.6 Hz, 1H), 5.71 (s, 1H), 3.99-3.89 (m, 3H), 2.98-2.78 (m, 2H), 1.98-1.82 (m, 2H), 1.73 (s, 6H), 1.50-1.30 (m, 11H). LC-MS (M+H)⁺=500.1.

### Example A5:2-(8-fluoro-6-(5-fluoro-2-(piperidin-4-ylamino)pyrimidin-4-yl) quinolin-4-yl) propan-2-ol

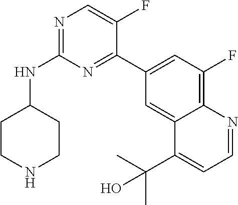 [SMILES: CC(C)(O)c1ccnc2c(F)cc(-c3nc(NC4CCNCC4)ncc3F)cc12]

A mixture of tert-butyl 4-({5-fluoro-4-[8-fluoro-4-(2-hydroxypropan-2-yl) quinolin-6-yl]pyrimidin-2-yl}amino) piperidine-1-carboxylate (100 mg, 0.200 mmol) and trifluoroacetic acid (5 mL) in dichloromethane (5 mL) was stirred for 2 h at room temperature before concentrated under reduced pressure. The residue was purified by prep-HPLC under the following conditions: column, XBridge Shield RP18 OBD Column, 30×150 mm, 5 μm; mobile phase, acetonitrile in water (with 10 mmol/L ammonium bicarbonate and 0.1% ammonium hydroxide), 18% to 48% gradient in 9 min; detector, UV 254 nm. The title compound (23 mg, 29%) was obtained. ¹H NMR (300 MHz, DMSO-d6) δ 9.45 (s, 1H), 8.96 (d, J=4.6 Hz, 1H), 8.49 (d, J=3.8 Hz, 1H), 8.12-8.01 (m, 1H), 7.72 (d, J=4.6 Hz, 1H), 7.32 (d, J=7.7 Hz, 1H), 5.82-5.65 (m, 1H), 3.94-3.64 (m, 1H), 3.02-2.92 (m, 2H), 2.61-2.51 (m, 2H), 1.93-1.83 (m, 2H), 1.74 (s, 6H), 1.49-1.32 (m, 2H). LC-MS (M+H)⁺=400.1.

### Example A6 and A7: (3S,4R)-4-((4-(4-((S)-1-aminoethyl)-8-fluoroquinolin-6-yl)-5-fluoropyrimidin-2-yl)amino)tetrahydro-2H-pyran-3-ol & (3S,4R)-4-((4-(4-((R)-1-aminoethyl)-8-fluoroquinolin-6-yl)-5-fluoropyrimidin-2-yl)amino)tetrahydro-2H-pyran-3-ol

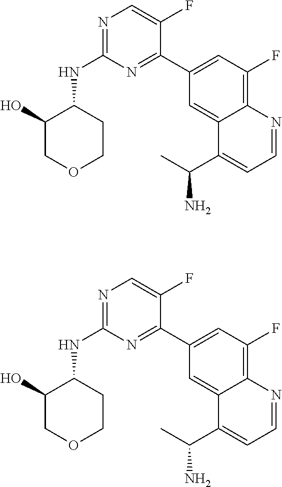 [SMILES: C[C@H](N)c1ccnc2c(F)cc(-c3nc(N[C@@H]4CCOC[C@H]4O)ncc3F)cc12 ; C[C@@H](N)c1ccnc2c(F)cc(-c3nc(N[C@@H]4CCOC[C@H]4O)ncc3F)cc12]

### Step 1:6-chloro-8-fluoroquinoline-4-carboxylic acid

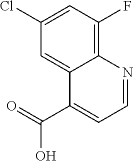 [SMILES: O=C(O)c1ccnc2c(F)cc(Cl)cc12]

A mixture of methyl 6-chloro-8-fluoroquinoline-4-carboxylate (5.2 g, 21.64 mmol) in tetrahydrofuran (40 mL) was added lithium hydroxide monohydrate (2.72 g, 64.92 mmol) in water (15 mL) dropwise at room temperature. The resulting mixture was stirred for 14 h at room temperature before concentrated under reduced pressure. The resulting mixture was diluted with water (50 mL) and extracted with dichloromethane (30 mL). The aqueous layer was acidified to pH 3 with 2 N HCl solution and extracted with ethyl acetate (2×60 mL). The organic phases were combined, washed with brine, dried over sodium sulfate, filtered and concentrated. The crude product (3.95 g, 81%) was used in the next step directly without further purification. LC-MS (M+H)⁺=226.0.

### Step 2:6-chloro-8-fluoro-N-methoxy-N-methylquinoline-4-carboxamide

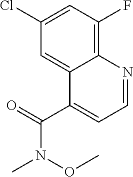 [SMILES: CON(C)C(=O)c1ccnc2c(F)cc(Cl)cc12]

To a mixture of 6-chloro-8-fluoroquinoline-4-carboxylic acid (350 mg, 1.550 mmol) and N,O-dimethylhydroxylamine hydrochloride (360 mg, 3.721 mmol) in acetonitrile (10 mL) were added diisopropylethylamine (180 mg, 1.397 mmol) and 1-(3-dimethylaminopropyl)-3-ethylcarbodiimide hydrochloride (725 mg, 4.651 mmol) in portions at room temperature under nitrogen atmosphere. The resulting mixture was stirred for 16 h at room temperature under nitrogen atmosphere before concentrated under reduced pressure. The resulting mixture was diluted with water (20 mL) and extracted with dichloromethane (20 mL×2). The organic phases were combined, washed with brine, dried over sodium sulfate, filtered and concentrated under reduced pressure. The residue was purified by flash chromatography eluting with ethyl acetate in petroleum ether (0-30% gradient, v/v) to yield the title compound (300 mg, 71%). LC-MS (M+H)⁺=269.0.

### Step 3:1-(6-chloro-8-fluoroquinolin-4-yl) ethan-1-one

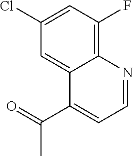

To a solution of 6-chloro-8-fluoro-N-methoxy-N-methylquinoline-4-carboxamide (300 mg, 1.115 mmol) in tetrahydrofuran (15.00 mL) was added methylmagnesium bromide (3M in diethyl ether, 0.560 mL, 1.67 mmol) dropwise at 0 °C under nitrogen atmosphere. The resulting mixture was stirred for 3 h at 0 °C under nitrogen atmosphere before quenched by addition of saturated ammonium chloride (20 mL). The resulting mixture was extracted with ethyl acetate (2×20 mL). The organic phases were combined, washed with brine, dried over sodium sulfate, filtered and concentrated. The residue was purified by flash chromatography eluting with ethyl acetate in petroleum ether (0-25% gradient, v/v) to yield the title compound (150 mg, 60%). LC-MS (M+H)⁺=224.0.

### Step 4: (S,Z)—N-(1-(6-chloro-8-fluoroquinolin-4-yl)ethylidene)-2-methylpropane-2-sulfinamide

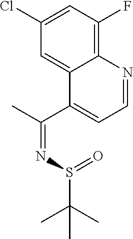 [SMILES: C/C(=N/[S@@](=O)C(C)(C)C)c1ccnc2c(F)cc(Cl)cc12]

To a mixture of 1-(6-chloro-8-fluoroquinolin-4-yl) ethanone (200 mg, 0.893 mmol) and(S)-2-methylpropane-2-sulfinamide (140 mg, 1.160 mmol) in tetrahydrofuran (10 mL) was added titanium (IV) ethoxide (408 mg, 1.787 mmol) dropwise at room temperature under nitrogen atmosphere. The resulting mixture was stirred for 16 h at 70 °C under nitrogen atmosphere. When the reaction was cooled to room temperature, the resulting mixture was concentrated under reduced pressure. The resulting mixture was diluted with water (15 mL) and extracted with ethyl acetate (25 mL×2). The organic phases were combined, washed with brine, dried over sodium sulfate, filtered and concentrated under reduced pressure. The residue was purified by flash chromatography eluting with ethyl acetate in petroleum ether (0-20% gradient, v/v) to yield the title compound (275 mg, 94%). LC-MS (M+H)⁺=327.0.

### Step 5: (S)—N-(1-(6-chloro-8-fluoroquinolin-4-yl)ethyl)-2-methylpropane-2-sulfinamide

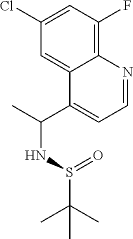 [SMILES: CC(N[S@@](=O)C(C)(C)C)c1ccnc2c(F)cc(Cl)cc12]

To a stirred solution of (S,Z)—N-(1-(6-chloro-8-fluoroquinolin-4-yl)ethylidene)-2-methylpropane-2-sulfinamide (275 mg, 0.841 mmol) in methanol (8 mL) was added sodium borohydride (95 mg, 2.511 mmol) in portions at 0 °C. The resulting mixture was stirred for 4 h at room temperature before quenched by the addition of icy water (20 mL). The resulting mixture was with ethyl acetate (2×20 mL). The organic phases were combined, washed with brine, dried over sodium sulfate, filtered and concentrated. Na₂SO₄. The residue was purified by flash chromatography eluting with methanol in dichloromethane (0% to 20% gradient, v/v) to yield the title compound (230 mg, 83%). LC-MS (M+H)⁺=329.0.

### Step 6: (4-(1-(((S)-tert-butylsulfinyl)amino)ethyl)-8-fluoroquinolin-6-yl) boronic acid

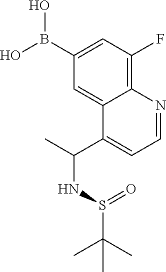 [SMILES: CC(N[S@@](=O)C(C)(C)C)c1ccnc2c(F)cc(B(O)O)cc12]

The title compound (288 mg, crude) was prepared in a manner similar to that in Example A1 step 5 from(S)—N-(1-(6-chloro-8-fluoroquinolin-4-yl)ethyl)-2-methylpropane-2-sulfinamide. The crude product was used in the next step directly without further purification. LC-MS (M+H)⁺=339.0.

### Step 7: (S)—N-(1-(6-(2-chloro-5-fluoropyrimidin-4-yl)-8-fluoroquinolin-4-yl)ethyl)-2-methylpropane-2-sulfinamide

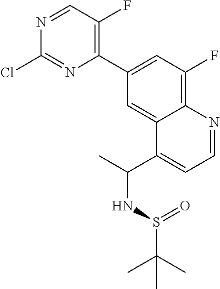 [SMILES: CC(N[S@@](=O)C(C)(C)C)c1ccnc2c(F)cc(-c3nc(Cl)ncc3F)cc12]

The title compound (230 mg, 73%, 2 steps) was prepared in a manner similar to that in Example A1 step 6 from (4-(1-(((S)-tert-butylsulfinyl)amino)ethyl)-8-fluoroquinolin-6-yl) boronic acid and 2,4-dichloro-5-fluoropyrimidine. LC-MS (M+Na)⁺=447.0.

### Step 8: (S)—N-(1-(8-fluoro-6-(5-fluoro-2-(((3S,4R)-3-hydroxytetrahydro-2H-pyran-4-yl)amino)pyrimidin-4-yl) quinolin-4-yl)ethyl)-2-methylpropane-2-sulfinamide

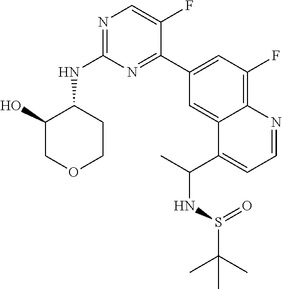 [SMILES: CC(N[S@@](=O)C(C)(C)C)c1ccnc2c(F)cc(-c3nc(N[C@@H]4CCOC[C@H]4O)ncc3F)cc12]

The title compound (175 mg, 63%) was prepared in a manner similar to that in Example A1 step 7 from(S)—N-(1-(6-(2-chloro-5-fluoropyrimidin-4-yl)-8-fluoroquinolin-4-yl)ethyl)-2-methylpropane-2-sulfinamide and (3S,4R)-4-aminooxan-3-ol hydrochloride. LC-MS (M+Na)⁺=528.2.

### Step 9: (3S,4R)-4-((4-(4-(1-aminoethyl)-8-fluoroquinolin-6-yl)-5-fluoropyrimidin-2-yl)amino)tetrahydro-2H-pyran-3-ol

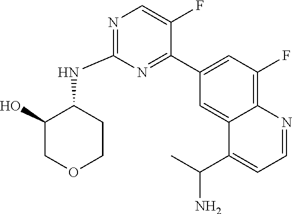 [SMILES: CC(N)c1ccnc2c(F)cc(-c3nc(N[C@@H]4CCOC[C@H]4O)ncc3F)cc12]

To a solution of(S)—N-(1-(8-fluoro-6-(5-fluoro-2-(((3S,4R)-3-hydroxytetrahydro-2H-pyran-4-yl)amino)pyrimidin-4-yl) quinolin-4-yl)ethyl)-2-methylpropane-2-sulfinamide (175 mg, 0.346 mmol) in methanol (1.5 mL) was added hydrochloride in methanol (4 M, 0.50 mL, 2.00 mmol) dropwise at room temperature under nitrogen atmosphere. The resulting mixture was stirred for 2 h at room temperature under nitrogen atmosphere before concentrated under reduced pressure. The resulting mixture was diluted with saturated sodium bicarbonate solution (15 mL). The resulting solution was extracted with ethyl acetate (2×15 mL). The organic phases were combined, washed with brine, dried over sodium sulfate, filtered and concentrated. The residue was purified by prep-HPLC under the following conditions: column, XBridge Shield RP18 OBD Column, 30×150 mm, 5 μm; mobile phase, acetonitrile in water (with 10 mmol/L ammonium bicarbonate and 0.1% ammonium hydroxide), 15% to 45% gradient in 8 min; detector, UV 254 nm. The title compound (80 mg, 57%) was obtained. LC-MS (M+H)⁺=402.1.

### Step 10: (3S,4R)-4-((4-(4-((S)-1-aminoethyl)-8-fluoroquinolin-6-yl)-5-fluoropyrimidin-2-yl)amino)tetrahydro-2H-pyran-3-ol & (3S,4R)-4-((4-(4-(R)-1-aminoethyl)-8-fluoroquinolin-6-yl)-5-fluoropyrimidin-2-yl)amino)tetrahydro-2H-pyran-3-ol

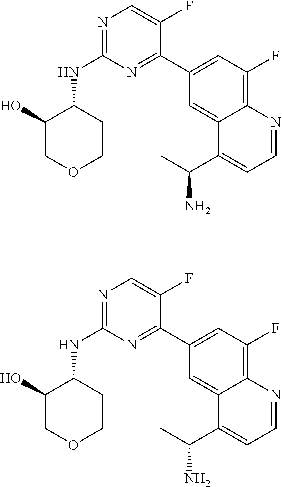 [SMILES: C[C@H](N)c1ccnc2c(F)cc(-c3nc(N[C@@H]4CCOC[C@H]4O)ncc3F)cc12 ; C[C@@H](N)c1ccnc2c(F)cc(-c3nc(N[C@@H]4CCOC[C@H]4O)ncc3F)cc12]

Example A7 and Example A8 were separated on chiral-HPLC to give (3S,4R)-4-((4-(4-((S)-1-aminoethyl)-8-fluoroquinolin-6-yl)-5-fluoropyrimidin-2-yl)amino)tetrahydro-2H-pyran-3-ol & (3S,4R)-4-((4-(4-((R)-1-aminoethyl)-8-fluoroquinolin-6-yl)-5-fluoropyrimidin-2-yl)amino)tetrahydro-2H-pyran-3-ol. Analytical chiral HPLC condition: CHIRALPAK IG, 2×25 cm, 5.0 μm. Mobile phase: (hexane: dichloromethane=3:1, containing 0.5% 2M ammonia in methanol): isopropanol, 20 mL/min in 26 min.

Example A7 (50 mg, 62%): ¹H NMR (400 MHz, DMSO-d6) δ 9.04-8.98 (m, 1H), 8.72 (s, 1H), 8.55-8.49 (m, 1H), 8.14-8.06 (m, 1H), 7.90 (d, J=4.6, 1H), 7.33 (d, J=7.8 Hz, 1H), 4.99-4.93 (m, 1H), 4.88-4.78 (m, 1H), 3.91-3.78 (m, 3H), 3.57-3.50 (m, 1H), 3.43-3.35 (m, 1H), 3.08 (t, J=10.4 Hz, 1H), 2.38-2.12 (br, 2H), 2.06-1.92 (m, 1H), 1.59-1.46 (m, 1H), 1.43 (d, J=6.6 Hz, 3H). LC-MS (M+H)⁺=402.2. Chiral HPLC: tR=3.949 min.

Example A8 (4 mg, 5%): ¹H NMR (400 MHz, DMSO-d6) δ 9.04-8.98 (m, 1H), 8.74 (s, 1H), 8.55-8.49 (m, 1H), 8.14-8.06 (m, 1H), 7.94-7.88 (m, 1H), 7.33 (d, J=7.8 Hz, 1H), 5.00-4.95 (m, 1H), 4.88-4.79 (m, 1H), 3.90-3.79 (m, 3H), 3.57-3.49 (m, 1H), 3.43-3.35 (m, 1H), 3.09 (t, J=10.3 Hz, 1H), 2.40-2.10 (m, 2H), 2.04-1.90 (m, 1H), 1.59-1.46 (m, 1H), 1.42 (d, J=6.7, 3H). LC-MS (M+H)⁺=402.2. Chiral HPLC: tR=5.594 min.

### Biological Assays

Compounds disclosed herein were tested for inhibition of CDK4/Cyclin D1 or CDK6/Cyclin D3 kinase in an assay based on the time-resolved fluorescence-resonance energy transfer (TR-FRET) methodology. The assay was carried out in 384-well low volume black plates in a reaction mixture containing CDK4/Cyclin D1 or CDK6/Cyclin D3, 1 mM ATP, 0.15 μM Rb (Ser780)-biotin substrate and 0-10 μM compound in buffer containing 50 mM HEPES pH7.0, 0.02% NaN3, 0.01% BSA, 0.1 mM Orthovanadate, 50 mM MgCl2, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK4/Cyclin D1 or CDK6/Cyclin D3 kinase activity in presence of increasing concentrations of compounds was calculated based on the ratio of fluorescence at 665 nm to that at 620 nm. The IC50 for each compound was derived from fitting the data to the four-parameter logistic equation by Dotmatics.

TABLE 1
Enzymatic activity IC50 (nM) for the compounds disclosed herein

| Example No. | CDK4D1 IC50 (nM) | CDK6D3 IC50 (nM) |
|---|---|---|
| A1 | 122 | 688 |
| A2 | 12 | 84 |
| A3 | 33 | 249 |
| A4 | 171 | 1700 |
| A5 | 83 | 220 |
| A6 | 32 | 760 |
| A7 | 97 | 2378 |

1. A compound of formula (I): 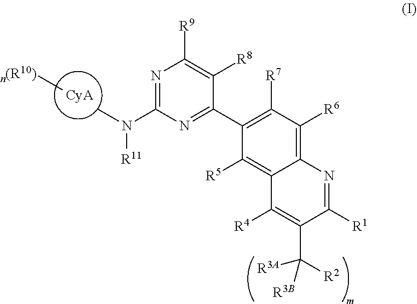
or a N-oxide thereof, or a pharmaceutically acceptable salt thereof, or a stereoisomer thereof, or a tautomer thereof, or a deuterated analog thereof, or a prodrug thereof,
wherein:
ring CyA is a 3- to 8-membered ring, said ring comprising 0, 1 or 2 heteroatom(s) independently selected from nitrogen, oxygen, or optionally oxidized sulfur as ring member(s); said ring is optionally substituted with at least one substituent R¹⁰;
n is 0, 1, 2, 3, 4 or 5;
m is 0 or 1; provided that when m is 0, the 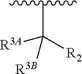
moiety as a whole is replaced with H;
R¹ is H, halogen, —C₁₋₈alkyl, —C₂₋₈alkenyl, —C₂₋₈alkynyl, cycloalkyl, haloalkyl, heterocyclyl, aryl, heteroaryl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1a}, R^{1b} and R^{1c} are each independently selected from hydrogen, —C₁₋₈alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f};
R^{1d} and R^{1f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R² is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2a}, —SO₂R^{2a}, —SO₂NR^{2a}R², —COR^{2a}, —CO₂R^{2a}, —CONR^{2a}R^{2b}, —NR^{2a}R²%, —NR^{2a}COR^{2b}, —NR^{2a}CO₂R^{2b}, —NR^{2a}CONR^{2b}R^{2c}, or —NR^{2a}SO₂R^{2b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2d};
R^{2a}, R^{2b} and R^{2c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(two R^{2d}) or (two R^{2f}) together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2g}, R^{2h} and R²ⁱ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3A} and R^{3B} are each independently hydrogen, halogen, —C₁₋₈alkyl, —C₂₋₈alkenyl, —C₂₋₈alkynyl, cycloalkyl, heterocyclyl, aryl, heteroaryl or —CN; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3c}; or
R^{3A} and R^{3B} together with the atom to which they are attached, form acyl (—C(═O)—) or a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3d}, R^{3e} and R^{3f} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁴ is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{401b}, —SO₂R^{401b}, —SO₂NR^{401b}R^{401c}, —COR^{401b}, —CO₂R^{401b}, —CONR^{401b}R^{401c}, —NO₂, —NR^{401b}R^{4c}, —NR^{401b}COR^{401b}, —NR^{401b}CO₂R^{401b}, —NR^{401b}CONR^{401c}R^{401d} or —NR^{401b}SO₂R^{401c}, wherein each of said —C₁₋₈alkyl, —C₂₋₈alkenyl, —C₂₋₈alkynyl, cycloalkyl heterocyclyl, aryl or heteroaryl is optionally substituted with at least one substituent R^{4a};
R^{401b}, R^{401c} and R^{401d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl;
R⁵, R⁶, R⁷, R⁸ and R⁹ are each independently selected from H, halogen, —C₁₋₈alkyl, —C₂₋₈alkenyl, —C₂₋₈alkynyl, cycloalkyl, heterocyclyl, aryl, heteroaryl, —CN, —OR^{5a}, —COR^{5a}, —CO₂R^{5a}, —CONR^{5a}R^{5b}, —NR^{5a}R⁵⁶, —NR^{5a}COR^{5b}, —NR^{5a}CO₂R^{5b} or —NR^{5a}CONR^{5b}R^{5c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5d};
R^{5a}, R^{5b} and R^{5c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5f};
R^{5d} and R^{5f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹⁰ is selected from H, halogen, —C₁₋₈alkyl, —C₂₋₈alkenyl, —C₂₋₈alkynyl, cycloalkyl, heterocyclyl, aryl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10d};
R^{10a}, R^{10b} and R^{10c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10f};
R^{10d} and R^{10f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R¹¹ is selected from H, —C₁₋₈alkyl, —C₂₋₈alkenyl, —C₂₋₈alkynyl; wherein each of said —C₁₋₈alkyl, —C₂₋₈alkenyl or —C₂₋₈alkynyl is optionally substituted with at least one substituent R^{11a}, and
R^{11a} is selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

2. The compound of claim 1, wherein the compound is selected from formula (IIa), (IIb), (IIc), (IId), (Ile) or (IIaa): 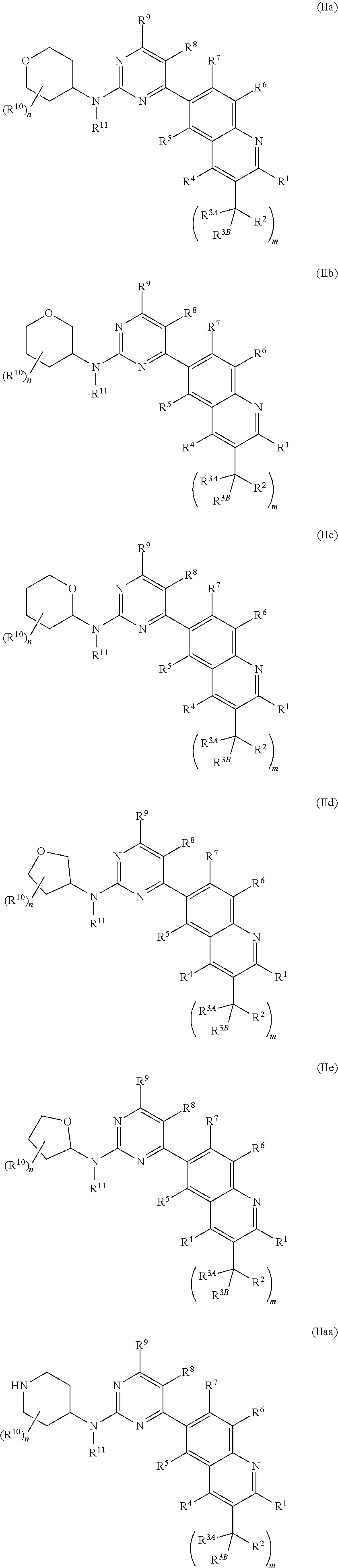
or a N-oxide thereof, or a pharmaceutically acceptable salt thereof, or a stereoisomer thereof, or a tautomer thereof, or a deuterated analog thereof, or a prodrug thereof.

3. The compound of claim 1, wherein ring CyA is a 3-, 4-, 5-, 6-, 7- or 8-membered ring, said ring comprising 0, 1 or 2 heteroatom(s) independently selected from nitrogen, oxygen, or optionally oxidized sulfur as ring member(s); said ring is optionally substituted with 0, 1, 2, 3, 4 or 5 R¹⁰; said ring is a saturated or unsaturated ring.

4. The compound of claim 1, wherein ring CyA is

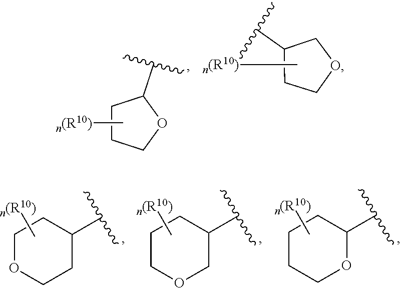

5. The compound of claim 1, wherein R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10d};
R^{10a}, R^{10b} and R^{10c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10f}; and
R^{10d} and R^{10f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

6. The compound of claim 1, wherein the 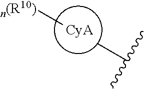
moiety is

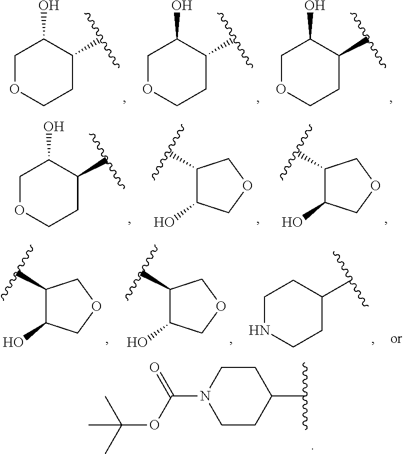

7. The compound of claim 1, wherein R¹ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl, heterocyclyl, —CN, —OR^{1a}, —COR^{1a}, —COR^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl or heterocyclyl is optionally substituted with at least one substituent R^{1d};
R^{1a}, R^{1b} and R^{1c} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl is optionally substituted with at least one substituent R^{1f}; and
R^{1d} and R^{1f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, balocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, clopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

8. The compound of claim 1, wherein R² is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2d};
R^{2a}, R^{2b} and R^{2c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2g}, —SO₂R²⁵, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) or (two R²¹) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; and
R^{2g}, R^{2h} and R²ⁱ are each independently selected from hydrogen, methyl, ethyl, propyl, butyl, pentyl, hexyl, beptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

9. The compound of claim 1, wherein R² is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2a}, —COR^{2a}, —CO₂R^{2a}, —CONR^{2a}R^{2b}, —NR^{2a}R^{2b}, —NR^{2a}COR^{2b}, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2e}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2g}, —SO₂R^{2g}, —COR^{2g}, —CO₂R^{2g}, —CONR^{2g}R^{2h}, —NO₂, —NR^{2g}R^{2h} or —NR^{2g}COR^{2h}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c] pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; and
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

10. The compound of claim 1, wherein R² is hydrogen, methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, —OR^{2a}, —NR^{2a}R^{2b} or —NR^{2a}COR^{2b}; wherein each of said methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl (n-propyl or iso-propyl), butyl (n-butyl, sec-butyl, iso-butyl or tert-butyl), pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c] pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2e}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f}; and
R^{2d} and R^{2f} are each independently selected from hydrogen, —F, —Cl, —Br, —I, methyl, ethyl, propyl, butyl, pentyl, —CF₃, —CF₂H, —CFH₂, —CH₂CF₃, —CF₂CH₃, —CH₂OH, —CH(CH₃)OH, —C(CH₃)₂OH, —CH₂CH₂OH, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OH, methoxy, ethoxy, propoxy, butoxy, pentoxy, hexoxy, hepthoxy, octoxy, —SO₂Me, —SO₂Et, —SO₂C₃H₇, —COMe, —COEt, —COC₃H₇, —NH₂, —NHCH₃, —N(CH₃)₂, —NHC₂H₅, —NHC₃H₇, —NHC₄H₉, —CONH₂, —CONHCH₃, —CON(CH₃)₂, —CONHC₂H₅, —CONHC₃H₇, —CONHC₄H₉.

11. The compound of claim 1, wherein R² is —H, -Me, —OMe, —OH, —NH₂, —NHCH₃, —N(CH₃)₂, —NHCH(CH₃)₂, —NHC(CH₃)₃, —NHCOCH₃,

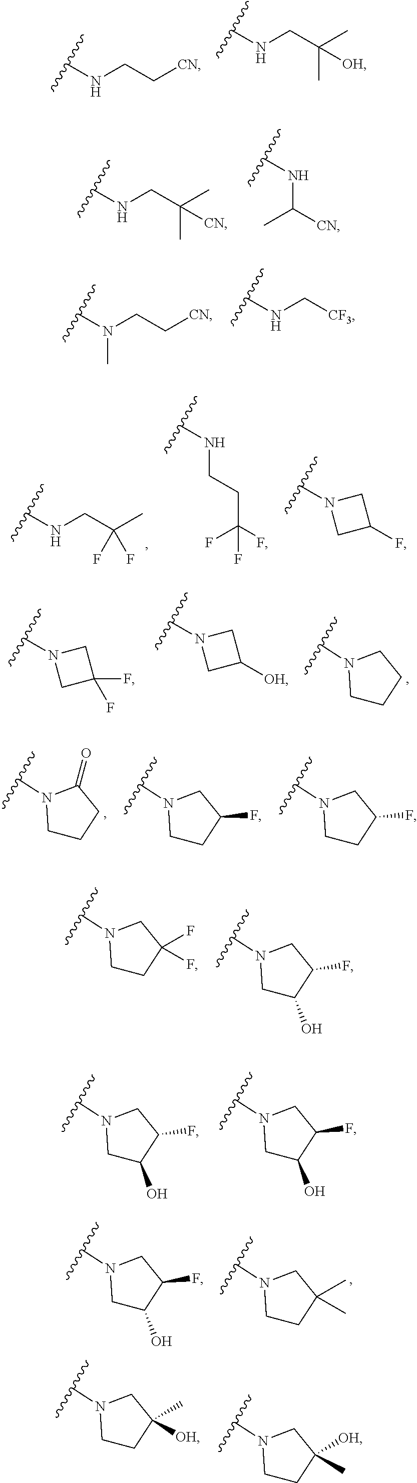

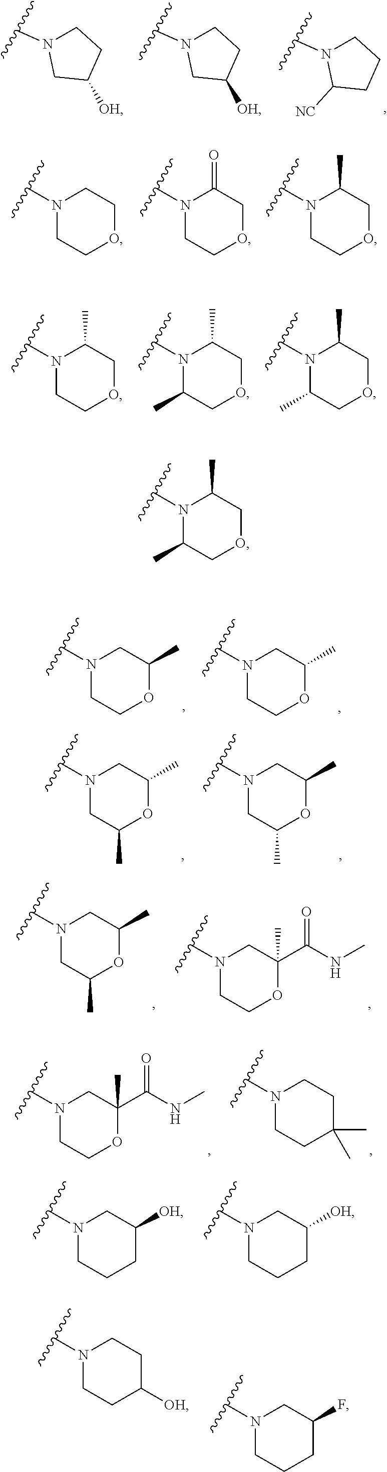

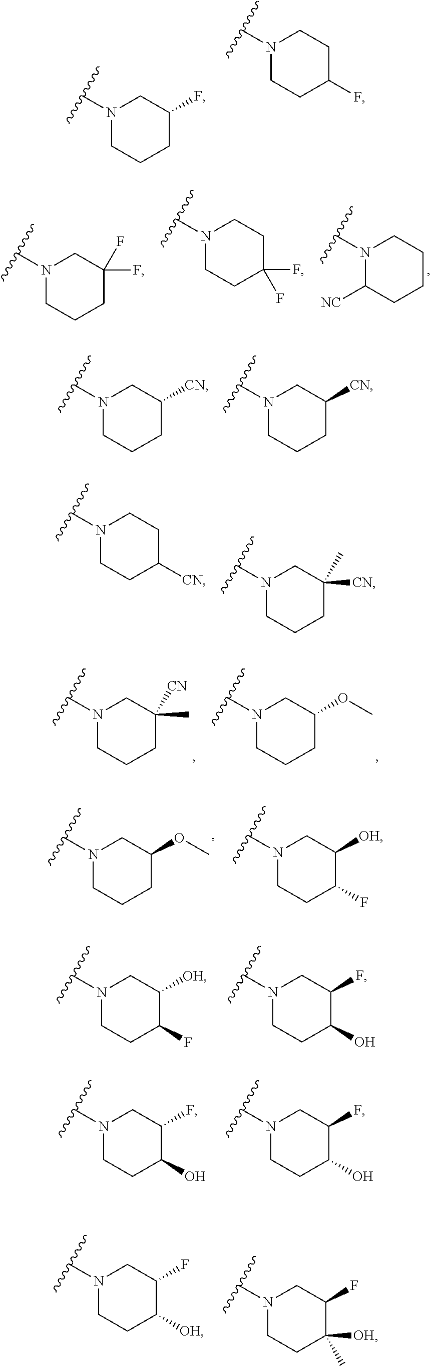

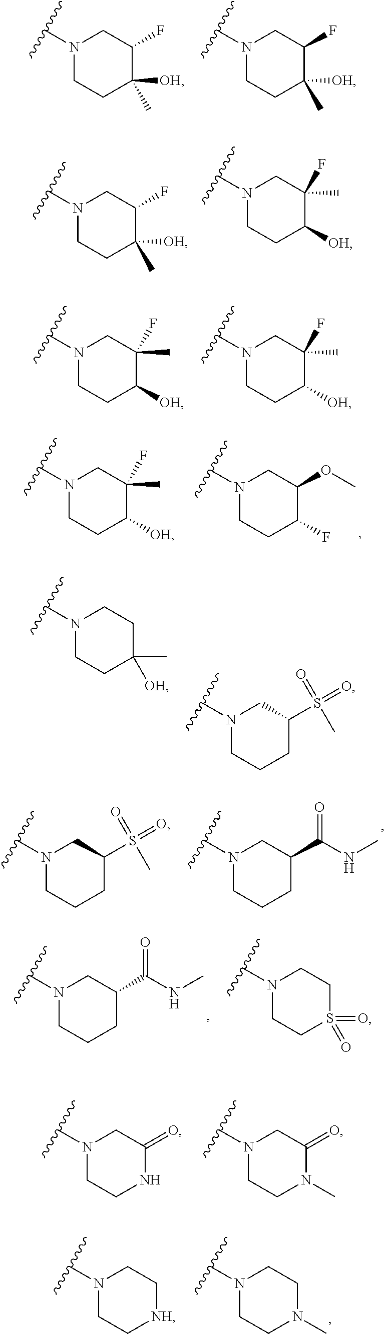

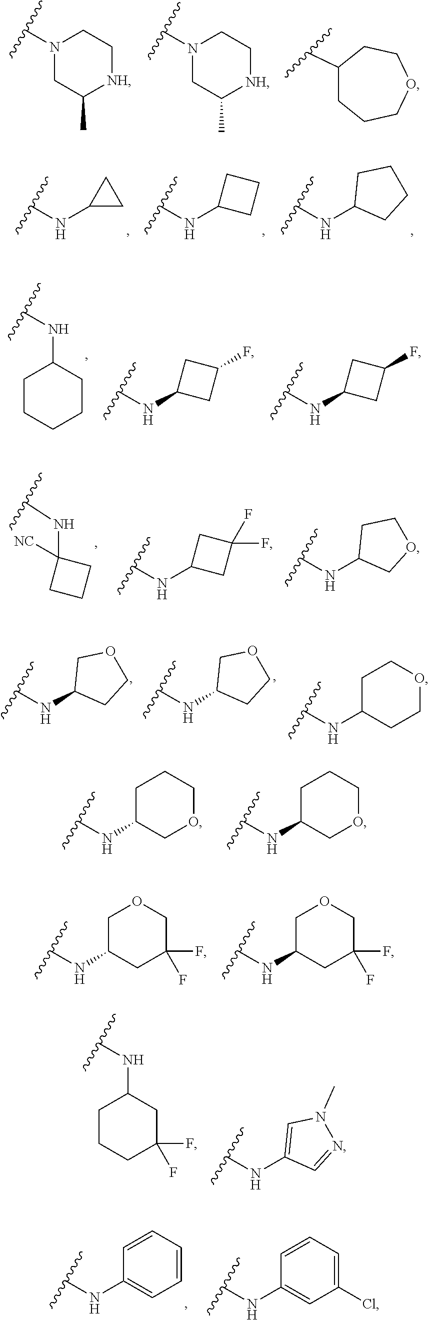

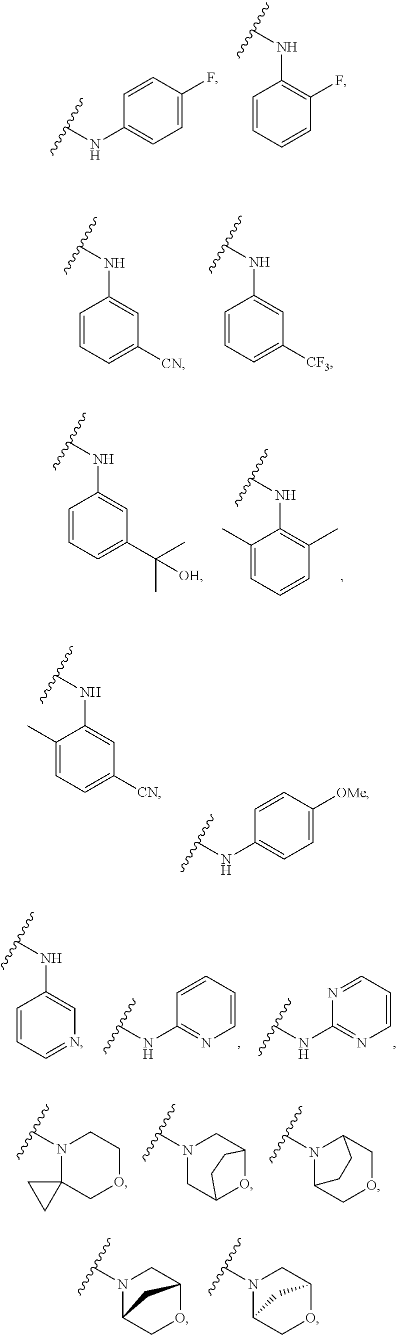

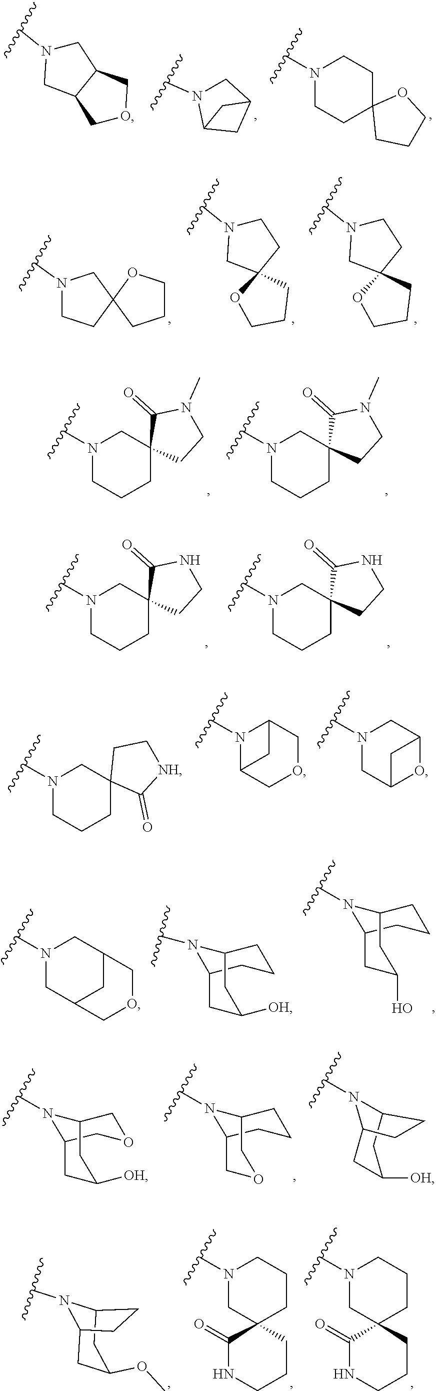

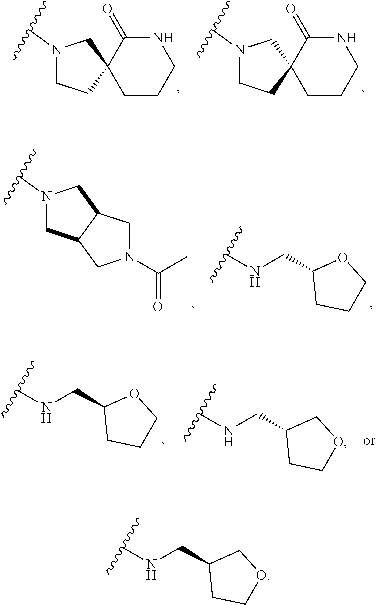

12. The compound of claim 1, wherein R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{3c}; or
R^{3A} and R^{3B} together with the atom to which they are attached, form acyl (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is mono-ring, spiro ring, fused ring or bridged ring, said ring is optionally substituted with at least one substituent R^{3c};
R^{3c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3d}R^{3f}, or —NR^{3d}SO₂R^{3e}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, —C₁₋₈alkoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; and
R^{3d}, R^{3e} and R^{3f} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl.

13. The compound of claim 1, wherein the 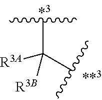
moiety is 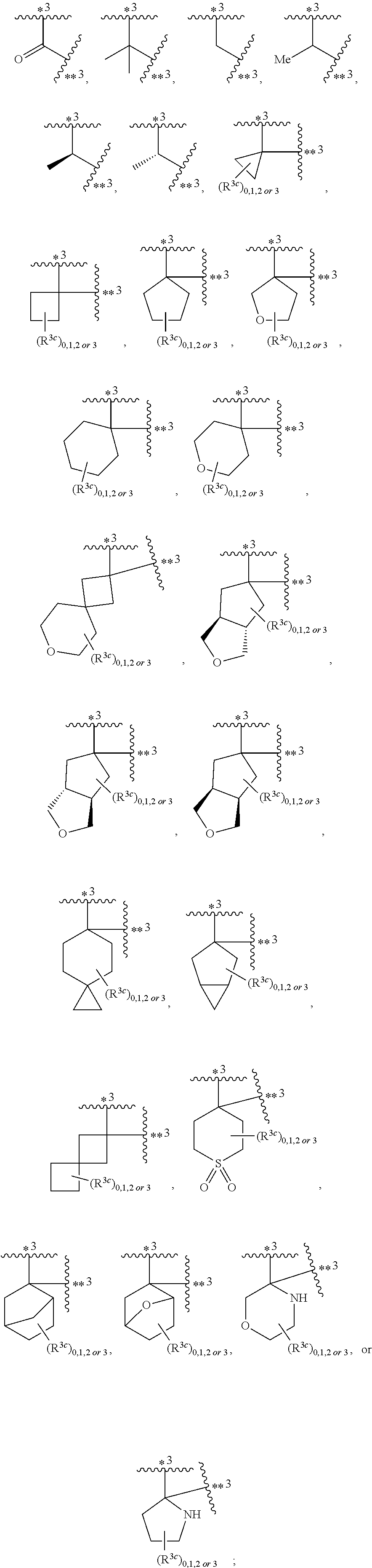
wherein *³ refers to the position attached to 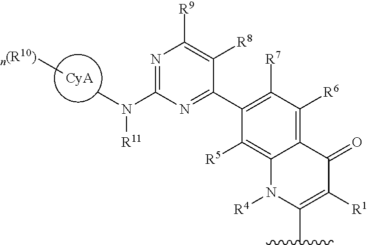
moiety, and **³ refers to the position attached to the 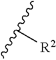
moiety.

14. The compound of claim 1, wherein the 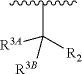
moiety is -Me, -Et,

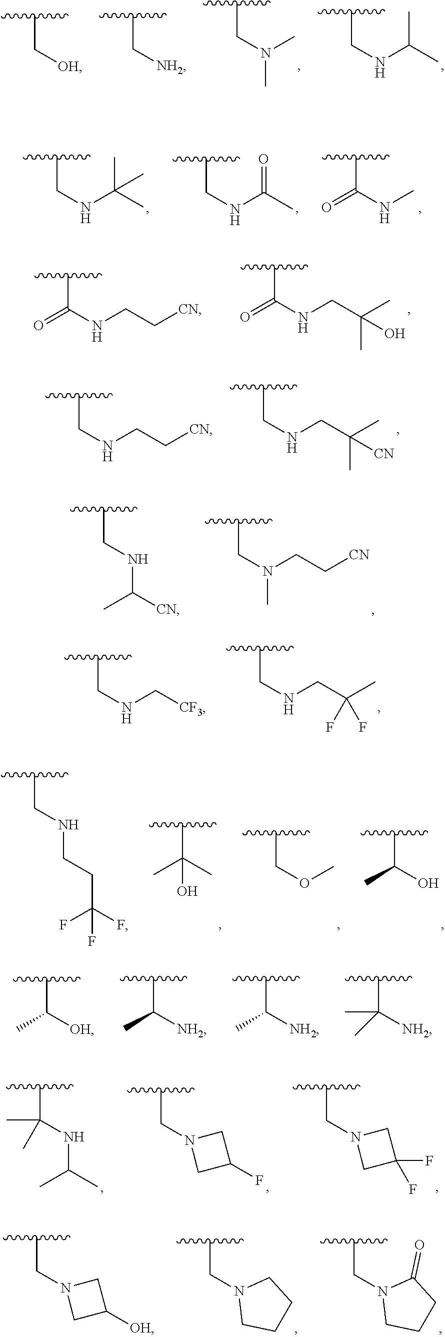

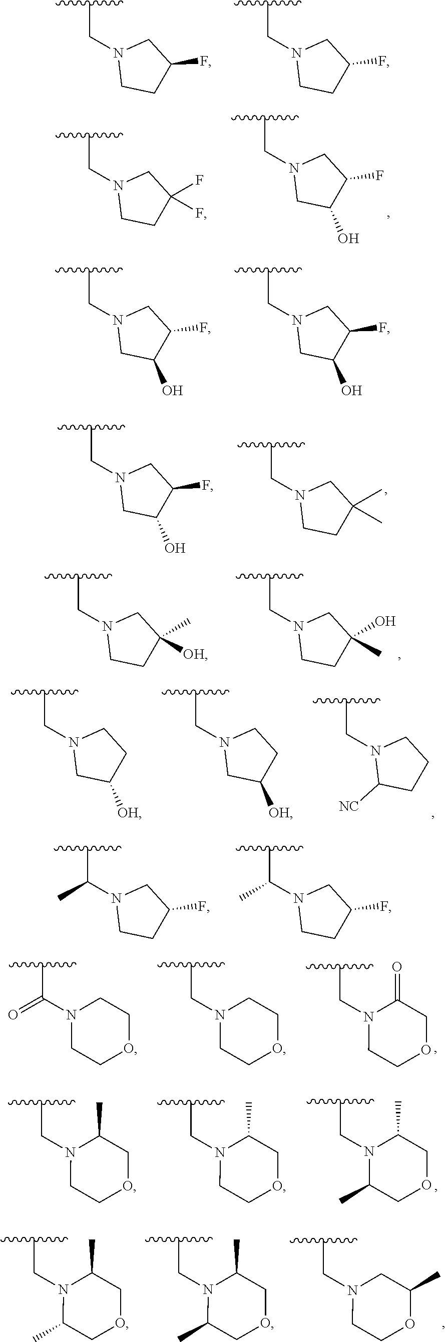

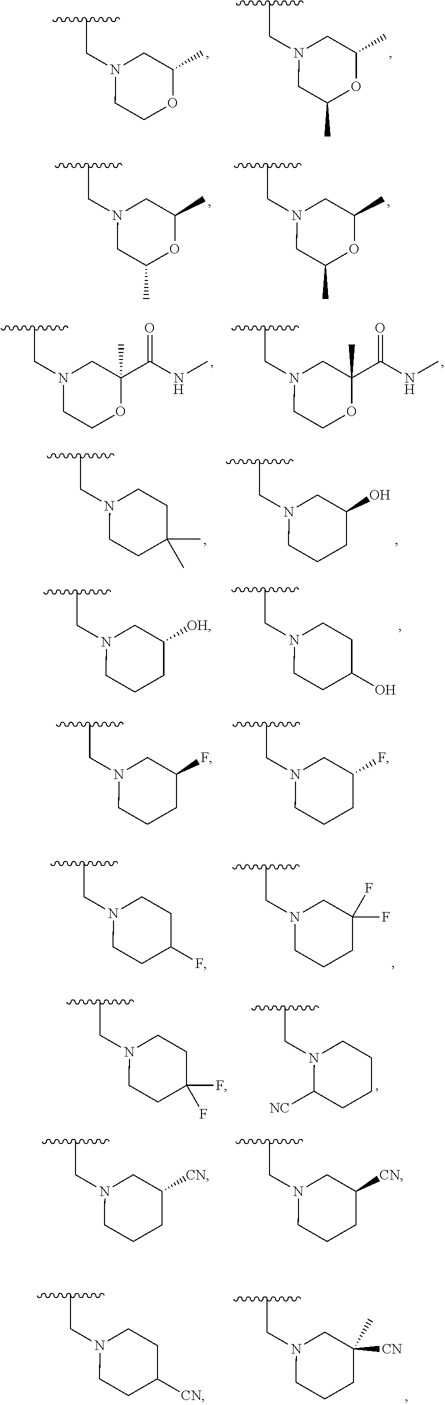

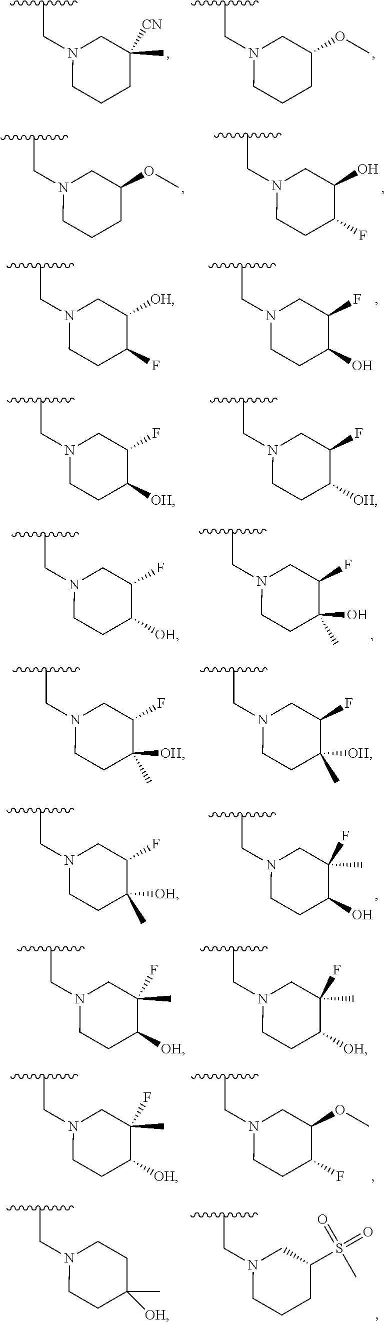

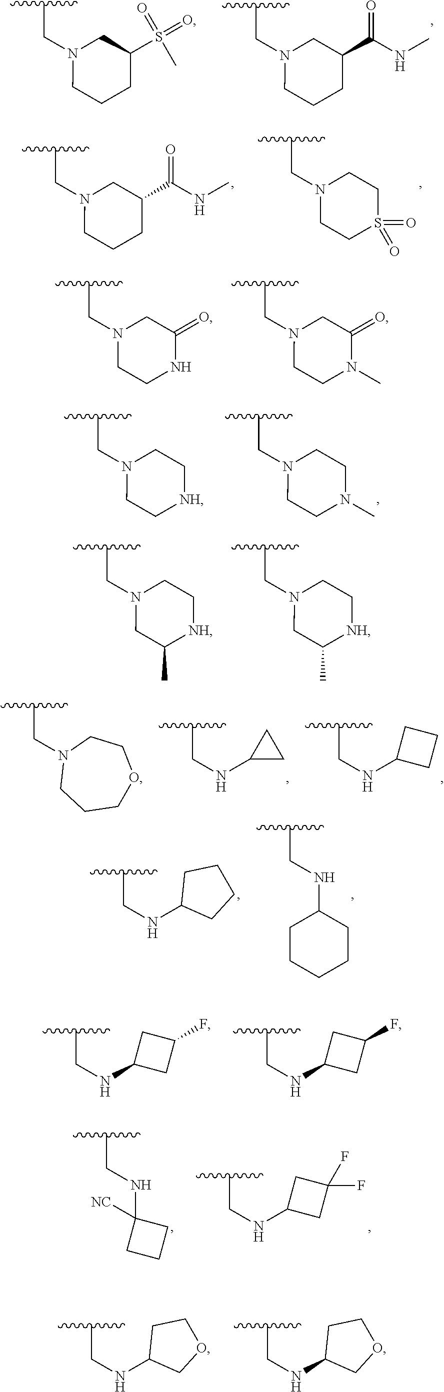

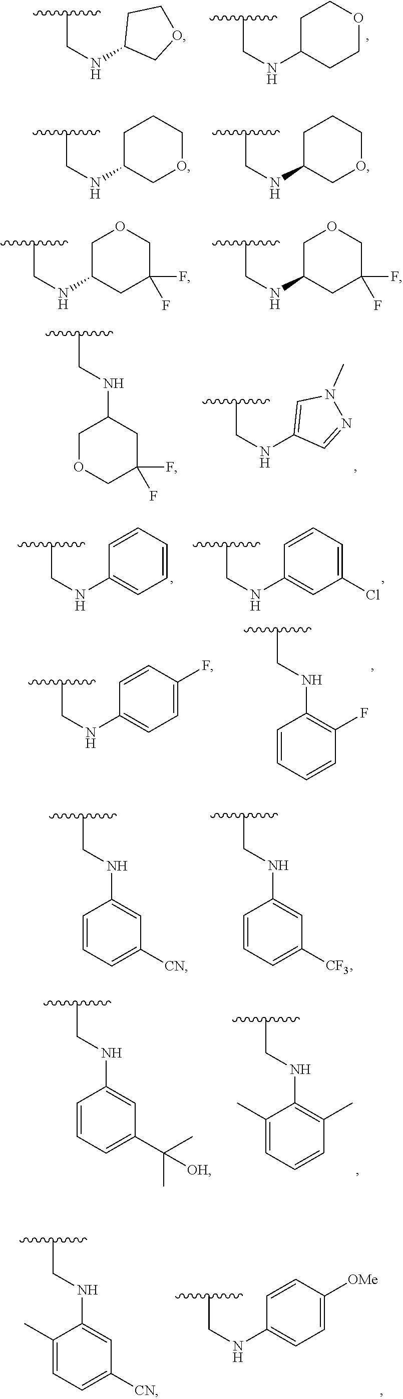

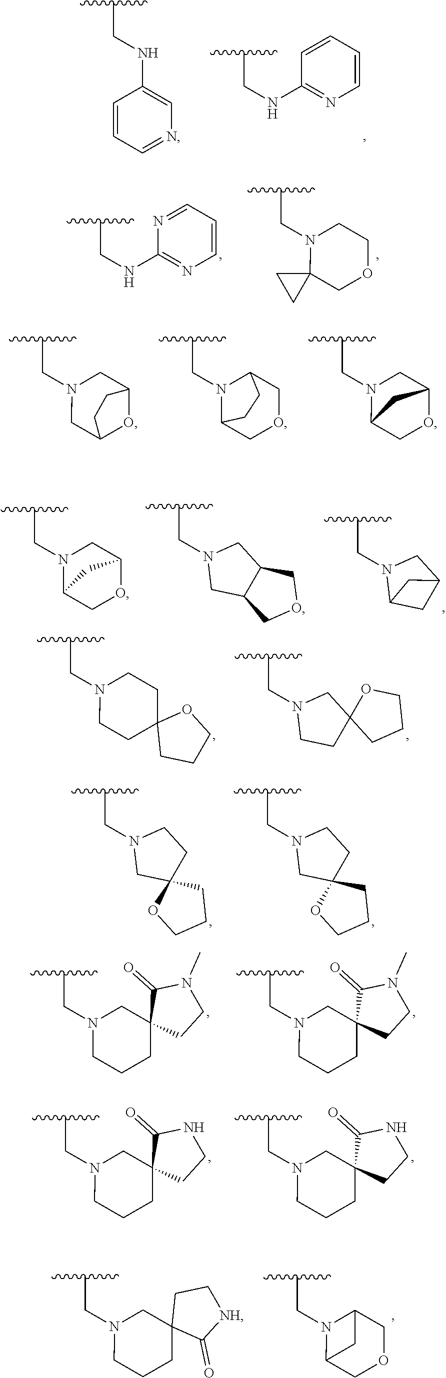

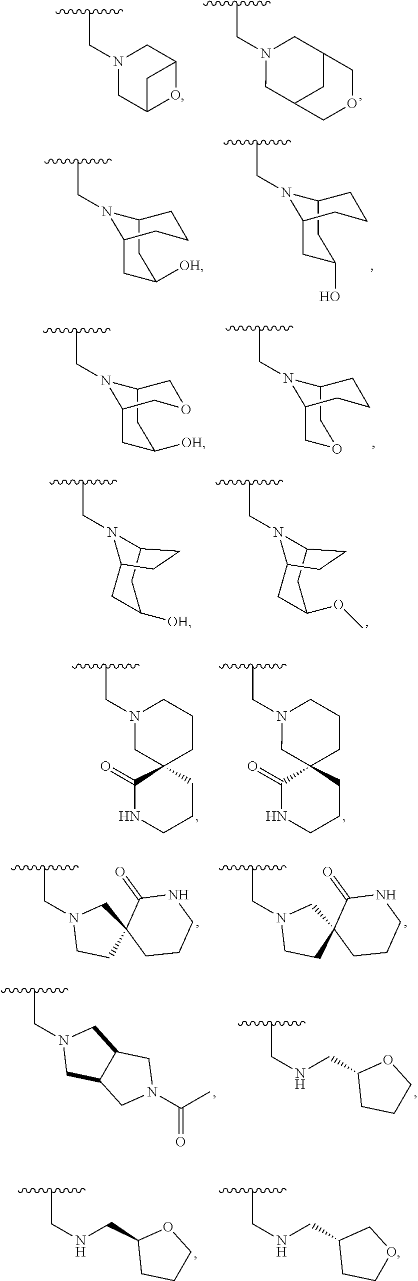

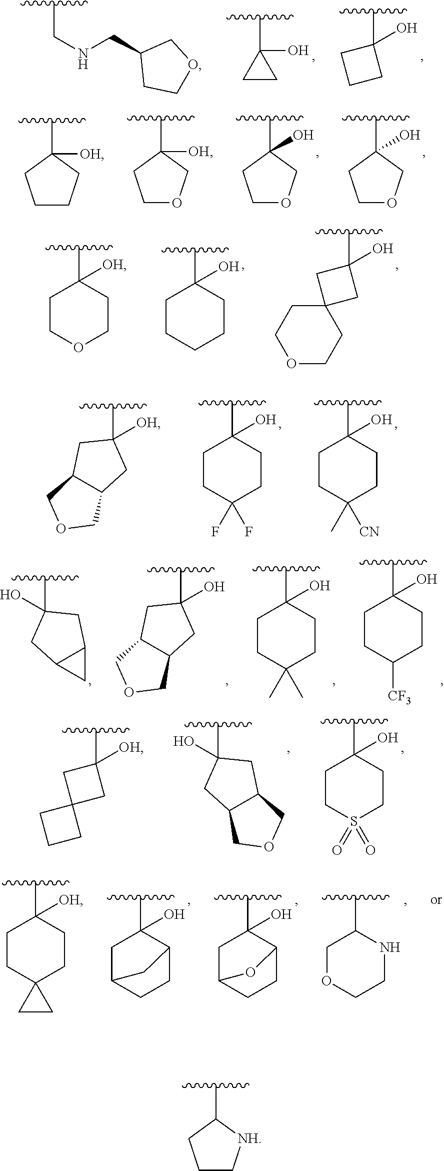

15. The compound of claim 1, wherein R⁴ is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{401b}, —SO₂R^{401b}, —SO₂NR^{401b}R^{401b}, —COR^{401b}, —CO₂R^{401b}, —CONR^{401b}R^{401b}, —NO₂, —NR^{401b}R⁴, —NR^{401b}COR^{401c}, —NR^{401b}CO₂R^{401c}, —NR^{401b}CONR^{401c}R^{401d} or —NR^{401b}SO₂R^{401c}, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent R^{4a}:
R^{401b}, R^{401c} and R^{401d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; and
R^{4b}, R^{4c} and R^{4d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

16. The compound of claim 1, wherein R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent Rod;
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5f}; and
R^{5d} and R^{5f} are each independently selected from hydrogen, —F, —Cl, —Br, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

17. The compound of claim 1, wherein R⁵, R⁶ and R⁷ are each independently selected from H, —F, —Cl, —Br, —I, methyl, ethyl, propyl, butyl, pentyl, hexyl, heptyl, octyl; and/or
R⁸ is selected from —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, hepthoxy, octoxy, —CN; and/or
R⁹ is selected from H.

18. The compound of claim 1, wherein R¹¹ is selected from H, methyl, ethyl, propyl, butyl, pentyl, hexyl, heptyl, octyl, —C₂₋₈alkenyl or —C₂₋₈alkynyl; wherein each of said methyl, ethyl, propyl, butyl, pentyl, hexyl, heptyl, octyl, —C₂₋₈alkenyl or —C₂₋₈alkynyl is optionally substituted with at least one substituent R^{11a}; and
R^{11a} is selected from hydrogen, halogen, hydroxy, —C₁₋₈alkyl, -haloC₁₋₈alkyl, —C₁₋₈alkoxy, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

19. The compound of claim 1, wherein m is 0, the 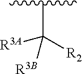
moiety as a whole is replaced with H.

20. The compound of claim 1, wherein the compound is selected from 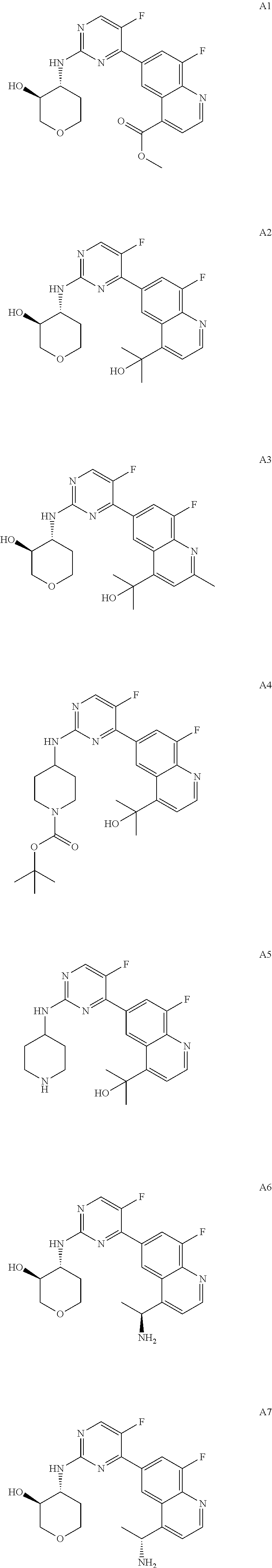
or a N-oxide thereof, or a pharmaceutically acceptable salt thereof, or a stereoisomer thereof, or a tautomer thereof, or a deuterated analog thereof, or a prodrug thereof.

21. A pharmaceutical composition comprising a compound of claim 1 or a N-oxide thereof, or a pharmaceutically acceptable salt thereof, or a stereoisomer thereof, or a tautomer thereof, or a deuterated analog thereof, or a prodrug thereof, together with a pharmaceutically acceptable excipient.

22. A method of decreasing CDK4 activity by inhibition, which comprises administering to an individual the compound according to claim 1, or a N-oxide thereof, or a pharmaceutically acceptable salt thereof, or a stereoisomer thereof, or a tautomer thereof, or a deuterated analog thereof.

23.-25. (canceled)

26. A method of treating a disease or disorder in a patient comprising administering to the patient a therapeutically effective amount of the compound of claim 1, or a N-oxide thereof, or a pharmaceutically acceptable salt thereof, or a stereoisomer thereof, or a tautomer thereof, or a deuterated analog thereof, wherein the disease or disorder is associated with inhibition of CDK4.

27. (canceled)
